(19) United States
(12) Patent Application Publication
(10) Pub. No.: US 2011/0023181 A1
(43) Pub. Date: Jan. 27, 2011

(54) Means for Improving Agrobiological Traits in a Plant by Providiing a Plant Cell Comprising in its Chloroplasts Enzymatic Activities for Converting Glycolate Into Malate

(75) Inventors: Véronica G. Maurino, Koln (DE); Ulf-Ingo Flügge, Koln (DE)

Correspondence Address: CONNOLLY BOVE LODGE & HUTZ, LLP, P O BOX 2207, WILMINGTON, DE, 19899, US

(73) Assignee: Universitat zu Koln, Koln (DE)

(21) Appl. No.: 12/918,637

(22) PCT Filed: Feb. 20, 2009
(86) PCT No.: PCT/EP2009/052023

§ 371 Date: Oct. 8, 2010

(30) Foreign Application Priority Data
Feb. 21, 2008 (EP) 08151759.1

(51) Int. Cl. C12N 15/82; C12N 5/04; C12N 5/10; A01H 5/00; A01H 5/10; C07H 21/00

(52) U.S. Cl. 800/278; 435/410; 435/419; 800/298; 536/23.2; 435/320.1

(57) ABSTRACT

The present invention is concerned with the improvement of aghcobiological traits in plants. More specifically, it relates to a plant cell comprising in its chloroplasts enzymatic activities for converting glycolate into malate. Preferably, said plant cell comprises a first polypeptide having glycolat oxidase activity, a second polypeptide having malate synthase activity and a third polypeptide having catalase activity or a first polypeptide having glycolat dehydrogenase activity and a second polypeptide having malate synthase activity. Also encompassed is a plant comprising said plant cells as well as seeds obtainable from the said plants. The present invention, furthermore, relates to a method for producing a transgenic plant or a plant cell having an increased water use efficiency or increased yield. The present invention contemplates polynucleotides comprising a combination of nucleic acids encoding the aforementioned polypeptides as well as vectors comprising the polynucleotides and uses thereof.

The present invention is concerned with the improvement of agricobiological traits in plants. More specifically, it relates to a plant cell comprising in its chloroplasts enzymatic activities for converting glycolate into malate. Preferably, said plant cell comprises a first polypeptide having glycolate oxidase activity, a second polypeptide having malate synthase activity and a third polypeptide having catalase activity or a first polypeptide having glycolate dehydrogenase activity and a second polypeptide having malate synthase activity. Also encompassed is a plant comprising said plant cells as well as seeds obtainable from the said plants. The present invention, furthermore, relates to a method for producing a transgenic plant or a plant cell having an increased water use efficiency or increased yield. The present invention contemplates polynucleotides comprising a combination of nucleic acids encoding the aforementioned polypeptides as well as vectors comprising the polynucleotides and uses thereof.

Photosynthetic CO₂ assimilation in C₃-plants is limited by environmental variables including temperature, CO₂ and water availability. Much of this limitation can be attributed to the catalytic properties of ribulose 1,5-bisphosphate carboxylase/oxygenase (RubisCO). Both atmospheric O₂ and O₂ produced by photosystem II compete with CO₂ for binding to the active site of RubisCO. The oxygenation of ribulose 1,5-bisphosphate (RuBP) yields only one molecule of glycerate 3-P, and the remaining two carbons form glycolate 2-P. This glycolate 2-P is the initial substrate of the C2-oxydative photosynthetic cycle (the photorespiratory cycle) leading to the loss of CO₂ [1].

The prime function of the photorespiratory cycle is to salvage glycolate 2-P (FIG. 1). In the course of this pathway, two molecules of glycolate 2-P are metabolised to form one molecule of each glycerate 3-P and CO₂, and these carbon compounds are used immediately for the regeneration of RuBP via the Calvin-Benson cycle (the C3-reductive photosynthetic cycle) without the net synthesis of triose phosphates [2, 3].

Glycolate 2-P is metabolized in three distinct cellular compartments namely chloroplasts, peroxisomes, and mitochondria (the site of the release of CO₂ and NH₄⁺), involving numerous enzymatic reactions and transport processes (FIG. 1). The photorespiratory pathway relies on the close integration of carbon and nitrogen metabolism in the leaf, because NH₄⁺ is released at the same rate as CO₂ [4]. Since nitrogen is a more valuable resource than carbon, the reassimilation of photorespired NH₄⁺ is essential for maintaining the nitrogen status in C₃-plants [5]. Ammonia released in the matrix of mitochondria during the course of glycine oxidation is used in the chloroplast by the glutamine synthetase (GS) catalysing the ATP-dependent conversion of glutamate to glutamine. Ferredoxin-dependent glutamate synthase (GOGAT), exclusively located to the chloroplasts of mesophyll cells, catalyses the conversion of glutamine and 2-oxoglutarate to two molecules of glutamate (FIG. 1).

One approach to improve the carboxylation activity of RubisCO would be to alter the kinetic constants of RubisCO for CO₂ and O₂. However, the genetic engineering of RubisCO by site-directed mutagenesis to decrease its oxygenase activity or its replacement with more efficient forms, like those from rhodophyte algae in which the CO₂/O₂ specificity factor is higher than that of the higher plant RubisCO, was so far only of limited success [6]. Another approach to improve C3 carbon fixation would be to reduce photorespiration directly by manipulation of enzymes in this pathway. From the analysis of insertional mutants it was demonstrated that blocking the photorespiratory metabolism downstream of RubisCO oxygenation is lethal [7-13]. These results suggest that photosynthesis is unlikely to be improved by direct manipulation of levels of enzymes in the photorespiratory pathway. On the other hand, several modes to increase the carboxylase activity of RubisCO by enhancing the actual concentration of CO₂ at its site of action have evolved. Examples are the CO₂ concentrating mechanisms of cyanobacteria and algae [14-15] or the photosynthetic cycle of C₄-plants [16]. The latter provides a CO2 pump, which leads to increased CO₂/O₂ ratio at the site of RubisCO and thus results in a decreased oxygenase activity relative to the carboxylase activity. In C3-plants, RubisCO operates in vivo at about 25% of its Vmax while it is believed to operate at or close to its Vmax in C4-plants [16]. Moreover, the specific activities of RubisCO are higher in C4-plants as compared to C₃-plants; hence less RubisCO protein is required to achieve high rates of photosynthesis in C₄-plants. Consequently and due to the fact that RubisCO is a N-expensive enzyme (20-30% of total leaf N in C₃-plants, but only 6% in C₄-plants), C₄-plants display greater photosynthetic nitrogen use efficiency than C₃-plants [17]. Attempts were performed to transfer the advantages of C₄-photosynthesis like high photosynthetic capacity, rapid growth, and increased nitrogen- and water-use efficiencies by overexpression of C4-cycle enzymes in C₃-plants such as A. thaliana, potato, tobacco and rice [18-21]. Häusler et al. [21] have shown that the overexpression of C4-cycle genes in C₃-plants (potato and tobacco) leads to an attenuation of photorespiration but also to changes in the pattern of endogenous enzymes and contents of UV-protectants. Recently, an attenuation of the flux through photorespiration by completing the pathway with both glyoxylate carboligase and tartronate semialdehyde reductase from E. coli was shown [22]. The photorespiratory cycle is not only a wasteful process inevitably resulting from the kinetic properties of RubisCO. It may also be regarded as a protecting mechanism for C₃-plants exposed to high temperatures and drought. To mention are the generation of metabolites, such as glycine, serine or one-carbon units, which can be exported from the leaf or used in other metabolic pathways, e.g. glycine for the synthesis of glutathione [23,24] and the prevention of excessive over-reduction of the electron transport chain by dissipating light energy, in particular when C₃-plants are exposed to high light intensities or drought stress (photoprotection; [25, 26]).

There is a need for improving CO₂ assimilation in plants and, in particular in C₃-plants in order to improve agricobiological traits of crop plants. However, at the same time the aforementioned drawbacks shall be avoided.

Accordingly, the technical problem underlying the present invention could seen as the provision of means and methods for complying with the aforementioned needs. The technical problem is solved by the embodiments characterized in the claims and herein below.

Thus, the present invention relates to a plant cell comprising in its chloroplasts enzymatic activities for converting glycolate into malate.

The term “plant cell” as used herein encompasses cells from all types of plant tissue, i.e. from leaves, roots, stems, vascular tissue, stalks, calli, cotelydons, stalks, anthers, petioles, seeds or embryonic plant tissue. The host cell according to the present invention can be obtained from any monocot or dicot plant. Preferably, it can be obtained from a model plant, preferably Arabidopsis thaliana, or a crop plant selected from the group consisting of oilseed rape, evening primrose, hemp, thistle, peanut, canola, linseed, soybean, safflower, sunflower, borage, maize, wheat, rye, oats, rice, barley, cotton, cassava, pepper, solanaceae plants, preferably, potato, tobacco, eggplant or tomato, vicia species, pea, alfalfa, bushy plants (coffee, cacao, tea), salix species, trees (oil palm, coconut) and perennial grasses and fodder crops. Suitable methods for obtaining host cells from the aforementioned plants as well as conditions for culturing these cells are well known in the art. Preferably, the plant cell is a cell of a C₃-plant.

The term “enzymatic activities” refers, preferably, to enzymatic activities which are introduced into the chloroplasts by introducing one or more polypeptides conferring the said activities, i.e. being capable of converting glycolate into malate, preferably, via glyoxylate as an intermediate. The polypeptides referred to herein shall, therefore, be enzymes exhibiting the aforementioned enzymatic activities when present in a chloroplast. Preferred polypeptides conferring the enzymatic activities necessary for the conversion are disclosed elsewhere in this specification in detail. It is to be understood that the polypeptides referred to herein may also exhibit further biological activities. In order to safeguard the localization of the polypeptides conferring the aforementioned activities into the chloroplast, the polypeptide, preferably, comprises a chloroplast transit peptide. Suitable chloroplast transit peptides are known in the art and are described in Bruce 2000, Trends in Cell Biology 10, 440-447 or Kleffmann 2004, Curr. Biol. 14, 354-362.

The plant cell of the present invention comprises a chloroplast having an endogenous ribulose 1,5-bisphosphate carboxylase/oxygenase activity (RubisCO). Due to the enzymatic properties of RubisCO and their dependencies on the CO₂/O₂ partial pressures, only a limited CO₂ fixation is possible in an unmodified chloroplast. The chloroplast comprised by the plant cell of the present invention is, however, engineered as to allow for conversion of glycolate into malate in the chloroplast by introducing into the chloroplast enzymatic activities which are capable of converting glycolate into glyoxlyate and, subsequently, convert the glyoxylate into malate. By introducing these enzymatic activities into the chloroplast, a complete gylcolate catabolic cycle is established. As a result of this cycle, one molecule of glycolate is converted into two molecules of CO₂ and reducing power in the form of NADPH and NADH (glycolate+O₂+NAD+NADP→2 CO₂+NADH+NADPH+2H⁺+H₂O). In this way, the CO₂ is directly released within the chloroplast, thereby increasing the CO₂ partial pressure. This, in turn, reduces the oxygenase activity of RubisCO whereby an autoregulatory cycle is created. To this end, the plant cell, preferably, further comprises an NADP-malic enzyme and a pyruvate dehydrogenase. Preferably, the plant cell of the present invention, thus, has an increased efficiency of CO₂ assimilation compared to a plant cell lacking enzymatic activities for converting glycolate into malate.

Advantageously, as a result of the surprisingly increased CO₂ assimilation, the plant cell of the present invention allows for the generation of plants and, in particular, crop plants, having increased yield and an improved water use efficiency as will be disclosed in more detail elsewhere in this specification. By introducing the aforementioned enzymatic activities and, thus, a complete glycolate catabolic cycle into chloroplasts, CO₂ will be directly released and re-fixed within the chloroplast and thereby reduce the oxygenase activity of RubisCO by increasing the CO₂/O₂ ratio (FIG. 1).

In a preferred embodiment of the plant cell of the present invention, the chloroplasts of the plant cell comprises a first polypeptide having glycolate oxidase activity, a second polypeptide having malate synthase activity and a third polypeptide having catalase activity.

A glycolate oxidase as referred to herein shall be capable of converting glycolate into glyoxylate and water peroxide (E.C. 1.1.3.15). A malate synthase shall be capable of synthesising malate by introducing two C-units from Acetyl-CoA into glyoxylate (E.C. 4.1.3.2). A catalase refers to an enzyme being capable of converting water peroxide into water and oxygen (E.C. 1.11.1.6). Any enzyme capable of carrying out these conversions and syntheses can be introduced into the chloroplast according to the present invention. The aforementioned enzymatic activities can be determined by assays well known in the art and described, preferably, in the accompanying Examples below.

Preferably, said first polypeptide is, however, encoded by a first polynucleotide comprising a nucleic acid selected from the group consisting of:
a. a nucleic acid having a nucleotide sequence as shown in SEQ ID No: 1;
b. a nucleic acid encoding a polypeptide having an amino acid sequence as shown in SEQ ID No: 2;
c. a nucleic acid having a nucleotide sequence being at least 50% identical to the nucleotide sequence shown in SEQ ID No: 1 wherein said nucleic acid encodes a polypeptide having glycolate oxidase activity; and
d. a nucleic acid encoding a polypeptide having an amino acid sequence being at least 50% identical to the amino acid sequence shown in SEQ ID No:2, wherein said polypeptide has glycolate oxidase activity.

Said second polypeptide is, preferably, encoded by a second polynucleotide comprising a nucleic acid selected from the group consisting of:
a. a nucleic acid having a nucleotide sequence as shown in SEQ ID No: 3;
b. a nucleic acid encoding a polypeptide having an amino acid sequence as shown in SEQ ID No: 4;
c. a nucleic acid having a nucleotide sequence being at least 40% identical to the nucleotide sequence shown in SEQ ID No: 3 wherein said nucleic acid encodes a polypeptide having malate synthase activity; and
d. a nucleic acid encoding a polypeptide having an amino acid sequence being at least 40% identical to the amino acid sequence shown in SEQ ID No: 4, wherein said polypeptide has malate synthase activity.

The third polypeptide, preferably, is encoded by a third polynucleotide comprising a nucleic acid selected from the group consisting of:
a. a nucleic acid having a nucleotide sequence as shown in SEQ ID No: 5;
b. a nucleic acid encoding a polypeptide having an amino acid sequence as shown in SEQ ID No: 6;
c. a nucleic acid having a nucleotide sequence being at least 50% identical to the nucleotide sequence shown in SEQ ID No: 5 wherein said nucleic acid encodes a polypeptide having catalase activity; and
d. a nucleic acid encoding a polypeptide having an amino acid sequence being at least 50% identical to the amino acid sequence shown in SEQ ID No: 6, wherein said polypeptide has catalase activity.

The term “polynucleotide” as used herein refers to a linear or circular nucleic acid molecule. It encompasses DNA as well as RNA molecules. The polynucleotide of the present invention shall be provided, preferably, either as an isolated polynucleotide (i.e. isolated from its natural context) or in genetically modified form. The term encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides. The polynucleotide of the present invention is characterized in that it shall encode a polypeptide as referred to above. The polynucleotide, preferably, has a specific nucleotide sequence as mentioned above. Moreover, due to the degeneracy of the genetic code, polynucleotides are encompassed which encode a specific amino acid sequence as recited above.

Moreover, the term “polynucleotide” as used in accordance with the present invention further encompasses variants of the aforementioned specific polynucleotides. Said variants may represent orthologs, paralogs or other homologs of the polynucleotide of the present invention. The polynucleotide variants, preferably, comprise a nucleic acid sequence characterized in that the sequence can be derived from the aforementioned specific nucleic acid sequences by at least one nucleotide substitution, addition and/or deletion whereby the variant nucleic acid sequence shall still encode a polypeptide having the activity as specified above. Variants also encompass polynucleotides comprising a nucleic acid sequence which is capable of hybridizing to the aforementioned specific nucleic acid sequences, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6×sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 by (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of the polypeptides of the present invention. Conserved domains of the polypeptide of the present invention may be identified by a sequence comparison of the nucleic acid sequence of the polynucleotide or the amino acid sequence of the polypeptide of the present invention with sequences of other members of the enzyme families referred to in accordance with this invention. Oligonucleotides suitable as PCR primers as well as suitable PCR conditions are described in the accompanying Examples. As a template, DNA or cDNA from bacteria, fungi, plants or animals may be used. Further, variants include polynucleotides comprising nucleic acid sequences which are at least 70%, at least 75%, at least 80%, at least 85%, at least 90%, at least 95%, at least 98% or at least 99% identical to the specific nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the specific amino acid sequences referred to herein. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS, 5 1989: 151-153) or the programs Gap and BestFit (Needleman 1970, J. Mol. Biol. 48; 443-453 and Smith 198, Adv. Appl. Math. 2; 482-489), which are part of the GCG software packet from Genetics Computer Group, 575 Science Drive, Madison, Wis., USA 53711, version 1991, are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the aforementioned nucleic acid sequences is also encompassed as a polynucleotide of the present invention. The fragment shall encode a polypeptide which still has the activity as specified above. Accordingly, the polypeptide may comprise or consist of the domains of the polypeptide of the present invention conferring the said biological activity. A fragment as meant herein, preferably, comprises at least 50, at least 100, at least 250 or at least 500 consecutive nucleotides of any one of the aforementioned nucleic acid sequences or encodes an amino acid sequence comprising at least 20, at least 30, at least 50, at least 80, at least 100 or at least 150 consecutive amino acids of any one of the aforementioned amino acid sequences.

The polynucleotides of the present invention either essentially consist of the aforementioned nucleic acid sequences or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part peptide sequences for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and comprise FLAG-tags, 6-histidine-tags, MYC-tags and the like.

More preferably, the first, second and/or third polypeptide(s) referred to above are expressed from heterologous polynucleotides, i.e. from polynucleotides which have been either transiently, e.g., by using an expression vector, or stably, e.g., by using T- or P-DNA insertion, introduced into the plant cell. The term “heterologous” as used herein means that the polynucleotides do not occur naturally in the plant cell. The term, thus, encompasses modified or unmodified polynucleotides which are derived from different organisms or modified polynucleotides derived from the plant cell of the invention. It is to be understood that the heterologous polynucleotide shall either comprise expression control sequences which allow for expression in the plant cell or sequences which allow for integration of the heterologous polynucleotide at a locus in the genome of the plant cell where the expression of the heterologous polynucleotide will be governed by endogenous expression control sequences of the plant cell. Preferably, the heterologous polynucleotide comprises a nucleic acid having a nucleic acid sequence of the first, the second or the third polynucleotide as referred to before, i.e. a polynucleotide encoding a glyoxylate oxidase, a polynucleotide encoding malate synthase or a polynucleotide encoding a catalase, two of the said nucleic acids or all three of them. By introducing the aforementioned heterologous polynucleotides, transgenic plant cells are generated. Such transgenic plant cells may be obtained by transformation techniques as published, and cited, in: Plant Molecular Biology and Biotechnology (CRC Press, Boca Raton, Fla.), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225. Preferably, transgenic plants can be obtained by T-DNA-mediated or P-DNA-mediated transformation. Such vector systems are, as a rule, characterized in that they contain at least the vir genes, which are required for the Agrobacterium-mediated transformation, and the sequences which delimit the T- or P-DNA (T-DNA border or P-DNA border). Suitable vectors are described elsewhere in the specification in detail.

In another preferred embodiment of the plant cell of the present invention, the chloroplasts comprise a first polypeptide having glycolate dehydrogenase activity and a second polypeptide having malate synthase.

As used herein, glycolate dehydrogenase (EC 1.1.99.14) refers to an enzyme being capable of converting glycolate into glyoxylate but using NAD⁺ instead of H₂O₂ as oxidant.

Preferably, said first polypeptide is encoded by a first polynucleotide comprising a nucleic acid selected from the group consisting of:
a. a nucleic acid having a nucleotide sequence as shown in SEQ ID No: 7;
b. a nucleic acid encoding a polypeptide having an amino acid sequence as shown in SEQ ID No: 8;
c. a nucleic acid having a nucleotide sequence being at least 50% identical to the nucleotide sequence shown in SEQ ID No: 7 wherein said nucleic acid encodes a polypeptide having glycolate dehydrogenase activity; and
d. a nucleic acid encoding a polypeptide having an amino acid sequence being at least 50% identical to the amino acid sequence shown in SEQ ID No:8, wherein said polypeptide has glycolate dehydrogenase activity.

Said second polypeptide, preferably, encoded by a second polynucleotide comprising a nucleic acid selected from the group consisting of:
a. a nucleic acid having a nucleotide sequence as shown in SEQ ID No: 3;
b. a nucleic acid encoding a polypeptide having an amino acid sequence as shown in SEQ ID No: 4;
c. a nucleic acid having a nucleotide sequence being at least 40% identical to the nucleotide sequence shown in SEQ ID No: 3 wherein said nucleic acid encodes a polypeptide having malate synthase activity; and
d. a nucleic acid encoding a polypeptide having an amino acid sequence being at least 40% identical to the amino acid sequence shown in SEQ ID No: 4, wherein said polypeptide has malate synthase activity.

As set forth above, said first and/or second polypeptide are, preferably, expressed from heterologous polynucleotide(s). Preferably, the heterologous polynucleotide, in this case, comprises either a nucleic acid having a nucleic acid sequence of the first or the second polynucleotide referred to before, i.e. a polynucleotide encoding a glycolate dehydrogenase or a polynucleotide encoding malate synthase, or both of the said nucleic acids.

The present invention also relates to a plant comprising the plant cell of the present invention.

The plant of the present invention, preferably, develops from the plant cell of the present invention. Thus, it is envisaged that all plant cells of the plant are plant cells according to the present invention, i.e., comprise in their chloroplasts enzymatic activities for converting glycolate into malate. As set forth above for the plant cells of the invention, preferred plants of the present invention are selected from the group consisting of: model plants, preferably Arabidopsis thaliana, or crop plants, preferably, oillseed rape, evening primrose, hemp, thistle, peanut, canola, linseed, soybean, safflower, sunflower, borage, maize, wheat, rye, oats, rice, barley, cotton, cassava, pepper, solanaceae plants, preferably, potato, tobacco, eggplant or tomato, vicia species, pea, alfalfa, bushy plants (coffee, cacao, tea), salix species, trees (oil palm, coconut) and perennial grasses and fodder crops. Preferably, the plant of the present invention is a C₃ plant.

Preferably, the plant of the present invention has increased CO₂ assimilation compared to a plant lacking a plant cell of the present invention. CO₂ assimilation as used herein refers to the conversion of CO₂ and water into organic molecules during photosynthesis. The chemical reactions in this context are well known in the art and are described in standard biochemical text books. An increase in CO₂ assimilation can be determined by techniques well known in the art and described in, preferably, in the accompanying Examples below. Preferably, the increase is statistically significant. Whether an increase is statistically significant can be determined by well known statistical tests including, e.g., Student's t-test, Mann-Whitney test etc. . . . More preferably, said increase is an increase of at least 10%, at least 20%, at least 30%, at least 40% or at least 50% in assimilated CO₂. A plant lacking a plant cell of the present invention as meant herein, preferably, refers to an unmodified control plant of the same variety as the plant of the present invention.

Also preferably, the plant of the present invention has an attenuated photorespiration compared to a plant lacking a plant cell of the present invention. Attenuation of the activity of the photorespiratory pathway can be determined by well known techniques. Suitable assays are disclosed in preferably, in the accompanying Examples below. Attenuation as referred to herein is, preferably, a statistically significant attenuation.

The plant of the present invention, preferably, has increased water use efficiency compared to a plant lacking a plant cell of the present invention. The water use efficiency is increased since the stomata in the leaves can be kept closer as a result of the increased CO₂ assimilation. Accordingly, the loss of water by evaporation is reduced. An increase in water use efficiency can be determined by techniques well known in the art and described in, preferably, in the accompanying Examples below. Preferably, the increase is statistically significant. More preferably, said increase is an increase of at least 10%, at least 20%, at least 30%, at least 40% or at least 50%.

Preferably, the plant of the present invention produces an increased number of leaves compared to a plant lacking a plant cell of the present invention. The number of leaves may be either counted or determined based on the fresh or dry weight of the leaves. Preferably, the increase in the number of leaves is statistically significant. More preferably, said increase is an increase in the numbers of leaves of at least 10%, at least 20%, at least 30%, at least 40% or at least 50%.

Furthermore, the plant of the present invention has an increased yield compared to a plant lacking a plant cell of the present invention. The term “yield” as used herein encompasses an increase in biomass (fresh or dry weight) of a plant part or the entire plant, and particularly, harvestable parts of the plant. The increase in biomass may be aboveground or underground. An increase in biomass underground may be due to an increase in the biomass of plant parts, such as tubers, rhizomes, bulbs etc. Particularly preferred is an increase in any one or more of the following: increased root biomass, increased root volume, increased root number, increased root diameter and increased root length. The term increased yield also encompasses an increase in seed yield. An increase in seed yield includes: (i) increased total seed yield, which includes an increase in seed biomass (seed weight) and which may be an increase in the seed weight per plant or on an individual seed basis; (ii) increased number of flowers (“florets”) per panicle; (iii) increased number of filled seeds; (iv) increased seed size; (v) increased seed volume; (vi) increased individual seed area; (vii) increased individual seed length and/or width; (viii) increased harvest index, which is expressed as a ratio of the yield of harvestable parts, such as seeds, over the total biomass; (ix) increased fill rate, (which is the number of filled seeds divided by the total number of seeds and multiplied by 100); and (x) increased thousand kernel weight (TKW), which is extrapolated from the number of filled seeds counted and their total weight. An increased TKW may result from an increased seed size and/or seed weight. An increased TKW may result from an increase in embryo size and/or endosperm size. Preferably, the increase in yield is statistically significant. More preferably, said increase is an increase of at least 10%, at least 20%, at least 30%, at least 40% or at least 50% in yield.

Finally, because this engineered pathway is localized to the chloroplasts, CO₂ can directly be re-fixed and less ammonia is released. Thus, a lesser extent of re-assimilation of ammonia is required and an improvement in the nitrate use efficiency also will occur in the plant of the present invention.

The present invention encompasses, furthermore, a seed obtainable from a plant of the present invention. The seed of the present invention shall be preferably capable of generating (i.e. developing into) a plant of the present invention and, specifically, a plant having the traits as defined above.

Plants or plant cells of the present invention are, preferably, transgenic plants or plant cells. Accordingly, the present invention contemplates a method for producing a transgenic plant or a plant cell having an increased water use efficiency compared to a corresponding non-transgenic plant or plant cell, said method comprises introducing in the chloroplasts of the transgenic plant or plant cell a first polypeptide having glycolate oxidase activity, a second polypeptide having malate synthase activity and a third polypeptide having catalase activity or a first polypeptide having glycolate dehydrogenase activity and a second polypeptide having malate synthase activity as defined above.

The introduction of the polypeptides is, preferably, achieved by introducing heterologous polynucleotides encoding the aforementioned polypeptides as discussed elsewhere in this specification in more detail. This includes transient introduction in expression vectors or stable integration into the genome of the plant cells via, e.g., T- or P-DNA insertion. It is to be understood that one heterologous polynucleotide comprising nucleic acids encoding the all of the aforementioned polypeptides may be introduced. Alternatively, separate heterologous polynucleotides each comprising a nucleic acid encoding a polypeptide of the aforementioned polypeptides may be introduced. Thus, the method, preferably, comprises the steps of:
a. introducing at least one heterologous polynucleotide encoding the said polypeptides into the plant of plant cell; and
b. expressing said polypeptides from the said at least one polynucleotide.

The present invention also contemplates a method for producing a transgenic plant or plant cell having increased yield compared to a corresponding non-transgenic plant or plant cell, said method comprises introducing in the chloroplasts of the transgenic plant or plant cell a first polypeptide having glycolate oxidase activity, a second polypeptide having malate synthase activity and a third polypeptide having catalase activity or a first polypeptide having glycolate dehydrogenase activity and a second polypeptide having malate synthase activity as defined above. The method, preferably, also comprises the steps of:
a. introducing at least one heterologous polynucleotide encoding the said polypeptides into the plant of plant cell; and
b. expressing said polypeptides from the said at laest one polynucleotide.

The present invention encompasses a polynucleotide comprising in combination a nucleic acid selected from the group consisting of:
a. a nucleic acid having a nucleotide sequence as shown in SEQ ID No: 1;
b. a nucleic acid encoding a polypeptide having an amino acid sequence as shown in SEQ ID No: 2;
c. a nucleic acid having a nucleotide sequence being at least 50% identical to the nucleotide sequence shown in SEQ ID No: 1 wherein said nucleic acid encodes a polypeptide having glycolate oxidase activity; and
d. a nucleic acid encoding a polypeptide having an amino acid sequence being at least 50% identical to the amino acid sequence shown in SEQ ID No:2, wherein said polypeptide has glycolate oxidase activity;

a nucleic acid selected from the group consisting of:
a. a nucleic acid having a nucleotide sequence as shown in SEQ ID No: 3;
b. a nucleic acid encoding a polypeptide having an amino acid sequence as shown in SEQ ID No: 4;
c. a nucleic acid having a nucleotide sequence being at least 40% identical to the nucleotide sequence shown in SEQ ID No: 3 wherein said nucleic acid encodes a polypeptide having malate synthase activity; and
d. a nucleic acid encoding a polypeptide having an amino acid sequence being at least 40% identical to the amino acid sequence shown in SEQ ID No: 4, wherein said polypeptide has malate synthase activity; and

a nucleic acid selected from the group consisting of:
a. a nucleic acid having a nucleotide sequence as shown in SEQ ID No: 5;
b. a nucleic acid encoding a polypeptide having an amino acid sequence as shown in SEQ ID No: 6;
c. a nucleic acid having a nucleotide sequence being at least 50% identical to the nucleotide sequence shown in SEQ ID No: 5 wherein said nucleic acid encodes a polypeptide having catalase activity; and
d. a nucleic acid encoding a polypeptide having an amino acid sequence being at least 50% identical to the amino acid sequence shown in SEQ ID No: 6, wherein said polypeptide has catalase activity.

The present invention, furthermore, relates to a polynucleotide comprising in combination

a nucleic acid selected from the group consisting of:
a. a nucleic acid having a nucleotide sequence as shown in SEQ ID No: 7;
b. a nucleic acid encoding a polypeptide having an amino acid sequence as shown in SEQ ID No: 8;
c. a nucleic acid having a nucleotide sequence being at least 50% identical to the nucleotide sequence shown in SEQ ID No: 7 wherein said nucleic acid encodes a polypeptide having glycolate dehydrogenase activity; and
d. a nucleic acid encoding a polypeptide having an amino acid sequence being at least 50% identical to the amino acid sequence shown in SEQ ID No:8, wherein said polypeptide has glycolate dehydrogenase activity; and

a nucleic acid selected from the group consisting of:
a. a nucleic acid having a nucleotide sequence as shown in SEQ ID No: 3;
b. a nucleic acid encoding a polypeptide having an amino acid sequence as shown in SEQ ID No: 4;
c. a nucleic acid having a nucleotide sequence being at least 40% identical to the nucleotide sequence shown in SEQ ID No: 3 wherein said nucleic acid encodes a polypeptide having malate synthase activity; and
d. a nucleic acid encoding a polypeptide having an amino acid sequence being at least 40% identical to the amino acid sequence shown in SEQ ID No: 4, wherein said polypeptide has malate synthase activity.

By introducing any one of the aforementioned polynucleotides of the present invention as a heterologous polynucleotide into a plant cell or plant, the traits referred to in accordance with the present invention will be conferred to the said plant or plant cell.

The present invention also contemplates a vector comprising one of the aforementioned polynucleotides of the present invention.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2^{nd} ed., Cold Spring Harbor Laboratory, Cold Spring Harbor Laboratory Press, Cold Spring Harbor, N.Y., 1989) and other laboratory manuals, such as Methods in Molecular Biology, 1995, Vol. 44, Agrobacterium protocols, Ed.: Gartland and Davey, Humana Press, Totowa, N.J. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T-DNA-mediated or P-DNA mediated transformation. Such vector systems are, as a rule, characterized in that they contain at least the vir genes, which are required for the Agrobacterium-mediated transformation, and the sequences which delimit the T-DNA or P-DNA (T-DNA or P-DNA borders). These vector systems, preferably, also comprise further cis-regulatory regions such as promoters and terminators and/or selection markers with which suitable transformed host cells or organisms can be identified. While co-integrated vector systems have vir genes and T-DNA sequences arranged on the same vector, binary systems are based on at least two vectors, one of which bears vir genes, but no T-DNA, while a second one bears T-DNA, but no vir gene. As a consequence, the last-mentioned vectors are relatively small, easy to manipulate and can be replicated both in E. coli and in Agrobacterium. These binary vectors include vectors from the pBIB-HYG, pCAMBIA, pPZP, pBecks, pGreen series. Preferably used in accordance with the invention is pGreen II. An overview of binary vectors and their use can be found in Hellens 2000, Trends in Plant Science 5, 446-451. Furthermore, by using appropriate cloning vectors, the polynucleotide of the invention can be introduced into host cells or organisms such as plants or animals and, thus, be used in the transformation of plants, such as those which are published, and cited, in: Plant Molecular Biology and Biotechnology (CRC Press, Boca Raton, Fla.), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225.

More preferably, the vector of the present invention is an expression vector. In such an expression vector, the polynucleotide comprises an expression cassette as specified above allowing for expression in eukaryotic cells or isolated fractions thereof. An expression vector may, in addition to the polynucleotide of the invention, also comprise further regulatory elements including transcriptional as well as translational enhancers. Preferably, the expression vector is also a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Suitable expression vector backbones are, preferably, derived from expression vectors known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith, D. B., and Johnson, K. S. (1988) Gene 67:31-40), pMAL (New England Biolabs, Beverly, Mass.) and pRIT5 (Pharmacia, Piscataway, N.J.), where glutathione S-transferase (GST), maltose E-binding protein and protein A, respectively, are fused with the nucleic acid of interest encoding a protein to be expressed. The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a coexpressed viral RNA polymerase (T7 gn1). This viral polymerase is provided by the host strains BL21 (DE3) or HMS174 (DE3) from a resident λ-prophage which harbors a T7 gn1 gene under the transcriptional control of the lacUV 5 promoter. Examples of vectors for expression in the yeast S. cerevisiae comprise pYeDesaturasec1 (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells (for example Sf9 cells) comprise the pAc series (Smith et al. (1983) Mol. Cell Biol. 3:2156-2165) and the pVL series (Lucklow and Summers (1989) Virology 170:31-39).

Expression vectors allowing expression in plant cells comprise those which are described in detail in: Becker, D., Kemper, E., Schell, J., and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, Ed.: Kung and R. Wu, Academic Press, 1993, p. 15-38. A plant expression cassette, preferably, comprises regulatory sequences which are capable of controlling the gene expression in plant cells and which are functionally linked so that each sequence can fulfill its function, such as transcriptional termination, for example polyadenylation signals. Preferred polyadenylation signals are those which are derived from Agrobacterium tumefaciens T-DNA, such as the gene 3 of the Ti plasmid pTiACH5, which is known as octopine synthase (Gielen et al., EMBO J. 3 (1984) 835 et seq.) or functional equivalents of these, but all other terminators which are functionally active in plants are also suitable. Since plant gene expression is very often not limited to transcriptional levels, a plant expression cassette preferably comprises other functionally linked sequences such as translation enhancers, for example the overdrive sequence, which comprises the 5′-untranslated tobacco mosaic virus leader sequence, which increases the protein/RNA ratio (Gallie et al., 1987, Nucl. Acids Research 15:8693-8711). Other preferred sequences for the use in functional linkage in plant gene expression cassettes are targeting sequences which are required for targeting the gene product into its relevant cell compartment. In the present case, in particular, the relevant compartment are the chloroplasts.

The present invention also relates to a composition comprising

a first polynucleotide comprising a nucleic acid selected from the group consisting of:
a. a nucleic acid having a nucleotide sequence as shown in SEQ ID No: 1;
b. a nucleic acid encoding a polypeptide having an amino acid sequence as shown in SEQ ID No: 2;
c. a nucleic acid having a nucleotide sequence being at least 50% identical to the nucleotide sequence shown in SEQ ID No: 1 wherein said nucleic acid encodes a polypeptide having glycolate oxidase activity; and
d. a nucleic acid encoding a polypeptide having an amino acid sequence being at least 50% identical to the amino acid sequence shown in SEQ ID No:2, wherein said polypeptide has glycolate oxidase activity;

a second polynucleotide comprising a nucleic acid selected from the group consisting of:
a. a nucleic acid having a nucleotide sequence as shown in SEQ ID No: 3;
b. a nucleic acid encoding a polypeptide having an amino acid sequence as shown in SEQ ID No: 4;
c. a nucleic acid having a nucleotide sequence being at least 40% identical to the nucleotide sequence shown in SEQ ID No: 3 wherein said nucleic acid encodes a polypeptide having malate synthase activity; and
d. a nucleic acid encoding a polypeptide having an amino acid sequence being at least 40% identical to the amino acid sequence shown in SEQ ID No: 4, wherein said polypeptide has malate synthase activity; and

a third polynucleotide comprising a nucleic acid selected from the group consisting of:
a. a nucleic acid having a nucleotide sequence as shown in SEQ ID No: 5;
b. a nucleic acid encoding a polypeptide having an amino acid sequence as shown in SEQ ID No: 6;
c. a nucleic acid having a nucleotide sequence being at least 50% identical to the nucleotide sequence shown in SEQ ID No: 5 wherein said nucleic acid encodes a polypeptide having catalase activity; and
d. a nucleic acid encoding a polypeptide having an amino acid sequence being at least 50% identical to the amino acid sequence shown in SEQ ID No: 6, wherein said polypeptide has catalase activity.

In addition, the present invention relates to a composition comprising a first polynucleotide comprising a nucleic acid selected from the group consisting of:
a. a nucleic acid having a nucleotide sequence as shown in SEQ ID No: 7;
b. a nucleic acid encoding a polypeptide having an amino acid sequence as shown in SEQ ID No: 8;
c. a nucleic acid having a nucleotide sequence being at least 50% identical to the nucleotide sequence shown in SEQ ID No: 7 wherein said nucleic acid encodes a polypeptide having glycolate dehydrogenase activity; and
d. a nucleic acid encoding a polypeptide having an amino acid sequence being at least 50% identical to the amino acid sequence shown in SEQ ID No:8, wherein said polypeptide has glycolate dehydrogenase activity; and

a second polynucleotide comprising a nucleic acid selected from the group consisting of:
a. a nucleic acid having a nucleotide sequence as shown in SEQ ID No: 3;
b. a nucleic acid encoding a polypeptide having an amino acid sequence as shown in SEQ ID No: 4;
c. a nucleic acid having a nucleotide sequence being at least 40% identical to the nucleotide sequence shown in SEQ ID No: 3 wherein said nucleic acid encodes a polypeptide having malate synthase activity; and
d. a nucleic acid encoding a polypeptide having an amino acid sequence being at least 40% identical to the amino acid sequence shown in SEQ ID No: 4, wherein said polypeptide has malate synthase activity.

As set forth elsewhere in this specification, such compositions comprises heterologous polynucleotides which can be used to confer the enzymatic activities to a plant or plant cell which are required for improving the traits referred to herein.

It will be understood that the aforementioned polynucleotides, vectors or compositions of the present invention are, preferably, to be used for conferring increased water use efficiency to a plant or plant cell or for conferring increased yield to a plant or plant cell.

### FIGURES

FIG. 1. The photorespiratory carbon and nitrogen cycle (black) of a C3-plant shortcircuited by the novel glycolate catabolic pathway (red). The transgenic enzymes introduced into A. thaliana chloroplasts are highlighted in green. Kat: catalase; GDC: glycine decarboxylase; GGAT: glutamate-glyoxylate aminotransferase; GK: glycerate kinase; GO: glycolate oxidase; GOGAT: glutamate-oxoglutarate aminotransferase; GS: glutamine synthetase; HPR: hydroxypyruvate reductase; MDH: NAD-malate dehydrogenase; ME: NADP-malic enzyme; MS: malate synthase; PDH: pyruvate dehydrogenase; PGP: phosphoglycolate phosphatase; SGAT: serine-glutamate aminotransferase; SHMT: serine hydroxymethyl transferase. Adapted from [3].

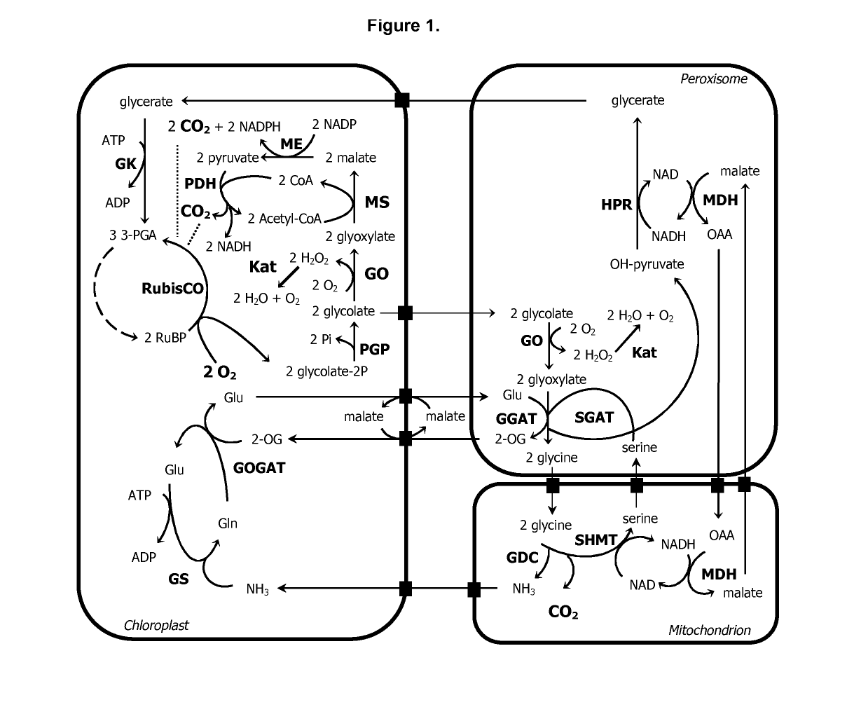

FIG. 2. Enzymatic activities in leaf extracts of transgenic and wild-type plants. A) Glycolate oxidase activity. B) Malate synthase activity. C) Catalase activity. Error bars indicate the SE of three different determinations.

FIG. 3. Growth characteristics of 6-week-old plants grown in short-day conditions at ambient (380 ppm) and high CO₂ (2,000 ppm) concentrations.

FIG. 4. Differences in the accumulation of starch in leaves of 6-week-old transgenic and wild-type plants grown in short-days (A-C); A) at 380 ppm; B) at 2,000 ppm; C) plants transferred for six days at 600 μmol quanta m−2 s−1 during the light period. D) Phenotype of the plants treated as in C.

The following examples merely illustrate the invention. They should, whatsoever, not be construed as limiting the scope of protection.

### EXAMPLES

### Example 1

### PCR Amplification of the Transgenes

The cDNAs corresponding to the glycolate oxidase 2 from A. thaliana leaves (GO, At3g14420) and the malate synthase from pumpkin cotyledons (MS, X56948) and the genomic sequence from E. coli catalase (KatE; M55161) were amplified using Platinum Pfx DNA polymerase (Invitrogen, Karlsruhe, Germany) and cloned into pGEMT-Easy (Promega, Mannheim, Germany) or alternatively, pCR-Blunt II-TOPO (Invitrogen). In the cases of GO and MS, the nucleotides encoding for the last amino acids (A/SRL), which presumably represent the peroxisomal targeting signal (31, 32), were omitted. The following primer combinations were used: GO fow (5′-TACAATTGGAGATCACTAACGTTACCGAGT-3′ SEQ ID NO: 9) and GO rev1 (5′-TGGGACACTCCACGTCCTTAGTCTAGACTAGTA-3′ SEQ ID NO: 10), MS fow (5′-ACACCGGTCGCTGGGAATGTATTCTGAATCGGCA-3′ SEQ ID NO: 11) and MS rev1 (5′-CACATAGGCATACATCATCCCAGGTGAGTCGACGTT-3′ SEQ ID NO: 12) KatE fow1 (5′-ACACCGGTCGCAACATAACGAAAAGAACCCA-3′SEQ ID NO: 13) and KatE rev1 (5′-ACGTCGACTCAGGCAGGAATTTTGTCAATCT-3′ SEQ ID NO: 14). To follow the cloning strategy, primers were designated to introduce unique MunI and SpeI sites at the 5′ and 3′ ends of GO and AgeI and SalI sites at the 5′ and 3′ ends of MS and KatE. In order to target GO to chloroplasts, the stromal targeting presequences from the A. thaliana phosphoglucomutase (PGM; 225 bp) was amplified by PCR using the following primers: PGM fow (5′-TAGGTACCCAATCAACAATGACGTCGACCTAC-3′ SEQ ID NO: 15) and PGM rev (5′-GAGATTAAATCGTTGCCGACGAAGCAATTGTA-3′ SEQ ID NO: 16).

The oligonucleotides were designated to introduce unique KpnI and MunI sites at the 5′ and 3′ends. The fragment obtained was cloned upstream the GO cDNA. To target MS and KatE to the chloroplast a fragment containing the tomato-RubisCO-small subunit (rbcS3C; X66072) promoter (715 bp) and transit peptide (172 bp) was amplified by PCR using genomic DNA and the following primers: rbcS3C fow (5′-ACGAGCTCATCCAGAATTGGCGTTGGATTA-3′ SEQ ID NO: 17) and rbcS3C rev (5′-AGCAACGGTGGAAGAGTCAGTTGCAACCGGTAT-3′ SEQ ID NO: 18). The primers were designated to introduce unique SacI and AgeI sites at the 5′ and 3′ ends. The fragment obtained was inserted upstream the MS and KatE coding regions. All plasmid constructions were sequenced using the PRISM fluorescent dye-terminator system (Applied Biosystems, Darmstadt, Germany) to exclude any possible mutation that could be created by the polymerase action.

### Example 2

### Construction of Binary Vectors

To direct the expression in A. thaliana, the DNAs encoding the plastidic precursor of the enzymes were cloned into a modified version of the binary vector pGreenII [33, 34]. The DNAs coding for the enzymes were cloned into the binary vector using different strategies. In the case of MS and KatE, the CaMV35S promoter was cut out of the vector and the tomato-rbcS3C promoter was used to direct the expression. The selection marker genes nosKan (kanamycin resistance), nosHyg (hygromycin resistance) and nosBAR 18 (BASTA resistance) were cloned into the StuI-site within the basic pGreenII vector. GO was cloned into the pGreenII 35S-nosKan vector, MS into the pGreenII rbcS3C-nosHyg vector and KatE into the pGreenII rbcS3C-nosBAR vector, the resulting plasmids were called 35S:GO, rbcS3C:MS and rbcS3C:KatE.

### Example 3

### Transformation of A. thaliana and Selection of Transformants

The binary vectors 35S:GO, rbcS3C:MS and rbcS3C:KatE were electroporated into Agrobacterium tumefaciens GV3101 bearing the helper plasmid pSoup and used to transform A. thaliana plants (Colombia ecotype, Col-0) via vacuum infiltration [35]. Transformed seeds were selected either by resistance to kanamycin, hygromycin or BASTA. Leaf material was collected from selected plants and DNA was extracted for PCR analyses. Plants containing the transgenes were allowed to self-pollinate. Transgenic lines were subjected to two more rounds of selection and characterization by means of PCR and enzymatic activity assays.

Plants were grown, in principle, under a 8 h-light/16 h-dark regime at photosynthetically active photon flux densities (PPFD) of 100 or 600 μmol quanta m-2 s-1 (normal and elevated light intensities, respectively) and adjusted to 22° C. day/18° C. night temperatures. For the growth at non-photorespiratory conditions, three-week-old plants grown in normal conditions were transferred to a chamber with a CO₂ concentration of 2,000±200 ppm.

### Example 4

### Production of GO-MS and GMK Transformants

GO-MS lines were produce by transformation of GO (line 5, GO5) with the vector rbcS3C:MS. The lines containing all three genes, GMK lines, were produced by retransformation of GO-MS (line 6, GO-MS6) with the vector rbcS3C:KatE. In all cases, the lines were selected taking advantage of the different resistances of the transgenes and corroborated by PCR, Southern blot and determination of enzymatic activities.

### Example 5

### Chloroplast Isolation, Preparation of Extracts and Enzymatic Measurements

Intact chloroplasts were isolated as described by Kebeish et al. [22]. Pelleted chloroplasts were resuspended in the corresponding extraction buffer (see below) and used directly for the enzymatic measurements. Glycolate oxidase: Leaf material was homogenized in the presence of N2 and resuspended in extraction buffer consisting in 100 mM Hepes, pH 7.2, 1 mM EDTA and 10 mM 2-mercaptoethanol. The homogenate was clarified by centrifugation and aliquots of 10 μl were used for the enzymatic assays following the method of Yamaguchi and Nishimura [36] with the following modifications: the reaction media contained 100 mM triethanolamine, pH 7.8, 3 mM EDTA, 0.75 mM oxidized glutathione and 4 mM phenylhydrazine. The reaction was started by the addition of 2.3 mM sodium glycolate and followed spectrophotometrically at 320 mm. One unit is defined as the amount of enzyme catalyzing the production of 1 μmol glyoxylate-phenylhydrazone per minute calculated from the extinction coefficient for phenylhydrazon at 324 nm of 16.8 mM−1 cm−1. Malate synthase: Leaf material was homogenized in the presence of N2 and resuspended in extraction buffer consisting in 50 mM Tris-HCl, pH 8.0 and 1 mM MgCl₂. The 20 homogenate was clarified by centrifugation and aliquots of 10 μl were used for the enzymatic assays. MS activity was determined following the procedure described by Smith et al. [37]. The reaction media contained 50 mM Tris-HCl, pH 8.0, 5 mM MgCl₂, 3 mM acetyl-CoA, 1,6 mM 5,5′-dithiobis 2-nitrobenzoic acid (DTNB). The reaction was started by the addition of 4 mM glyoxylate and followed spectrophotometrically at 410 mm at 25° C. One unit is defined as the amount of enzyme catalyzing the production of 1 μmol 4-nitrothiolate per minute calculated from the extinction coefficient for 4-nitrothiolate at 410 nm of 13.7 M−1 cm−1. Catalase: Leaf material was homogenized in the presence of N2 and resuspended in extraction buffer consisting in 50 mM KH₂PO₄, pH 7.0, 1% (v/v) polyvinylpyrrolidone (PVP)-40 and 0.1% (v/v) Triton X-100. Catalase activity was determined in 50 mM KH₂PO₄, pH 7.0 as described by Havir and McHale [38]. The reaction was started by the addition of 10 mM H₂O₂ and followed at 240 nm at 25° C. One unit is defined as the amount of enzyme catalyzing the decomposition of 1 μmol H₂O₂ per minute calculated from the extinction coefficient for H₂O₂ at 240 nm of 43.6 M−1 cm−1.

### Example 6

### Measurements of H₂O₂ and Glyoxylate Content

Hydrogen Peroxide: H₂O₂ content was determined following a modified version of the protocol described by Okuda et al. [39]. Leaves (50-100 mg) were homogenized in the presence of N₂ and 500 μl of 0.2 M HClO₄ and centrifuged at 20,000 g for 5 min at 4° C. To remove HClO₄, 300 μl of the supernatant was neutralized to pH 7.5 with 4 N KOH and the solution was centrifuged at 1,000 g for 1 min at 4° C. 200 μl of supernatant was applied to a 1.2 ml column of AG-1 (0.8 cm×4 cm; BioRad, Hercules, USA), and the column was washed two times with 800 μl of distilled water. The second eluate containing the H₂O₂ was used for the assay. The reaction mixture contained 500 μl of the eluate, 200 μl p-dimethylaminobenzalsehyde (DMAB, 12.5 mM in 0.375 M phosphate buffer, pH 6.5), 40 μl 3-methyl-2-benzothiazolinone hydrazone (MBTH, 0.05% w/v) and 10 μl peroxidase (12.5 U/ml) in a total volume of 750 μl. The reaction was started by the addition of peroxidase and after 8 min incubation at 25° C. the increase of absorbance at 590 nm was monitored. Glyoxylate: The determination of glyoxylate was conducted using a modification of the protocol described by Häusler et al. [40]. Leaf material (30-100 mg) was homogenized in the presence of N₂ and 500 μl 100 mM HCl and 0.1% phenylhydrazine and incubated at 80° C. for 5 min. After chilling on ice, the samples were centrifuged at 10,000 g for 2 min and 200 μl of the supernatant was mixed with 750 μl of 18.5% HCl and 50 μl of 4% (w/v) K₃Fe(CN)₆. The mixture was centrifuged at 10,000 g for 2 min and the absorbance of the supernatant was measured at 520 nm exactly 8 min after the addition of K₃Fe(CN)₆. As a control, K₃Fe(CN)₆ was omitted.

### Example 7

### Metabolite Contents

GC-MS analysis, carbohydrate and chlorophyll contents were conducted as described by Fahnenstich et al. [33].

### Example 8

### Chlorophyll Fluorescence Parameters

Chlorophyll fluorescence measurements were performed with a PAM-2000 pulse amplitude modulated chlorophyll fluorometer (Walz GmbH, Effeltrich, Germany). At the start of each measurement, a plant was dark-adapted for 10 min. Basal fluorescence (F0) was measured with modulated weak red light and maximal fluorescence (Fm) was induced with a saturating white light pulse (5000 μmol m−2 sec−1; duration 0.8 s).

### Example 9

### CO₂ Assimilation Rate and Carbon Isotope Analysis

Photosynthetic rate measurements were made on plants that were grown under a 8 hlight/16 h-dark regime at photosynthetically active photon flux densities (PPFD) of 100 and 300 μmol quanta m−2 s−1 (normal and elevated light intensities, respectively) at ambient relative humidity levels and at 24° C., following the procedures of Awada et al., (41). Briefly, the maximum net photosynthesis (A, μmol m−2 s−1) at light saturation were made using a portable photosynthetic system mounted with a LED light source and a leaf chamber specifically constructed for Arabidopsis leaves (LI 6400-2B, LICOR Inc., Lincoln, Nebr., USA) Carbon isotope ratio measurements were performed according to the procedures of Madhavan et al. [42]. For the determination of carbon isotope ratios, whole rosettes were dried at 60° C. for 48 h and ground to a fine powder. A sub-sample of this leaf powder (2-3 mg) was analyzed for carbon isotope ratio using an elemental analyzer (Heraeus, CHN—O Rapid) interfaced with an automated trapping box system and a Finnigan Delta-S isotope ratio mass spectrometer. The isotope ratio of each sample was determined by comparison with a previously calibrated working standard with a known δ13C value relative to PDB (Pee Dee Belemnite international standard).

### Example 10

### Co-Expression of Glycolate Oxidase, Malate Synthase and Catalase in A. thaliana Chloroplasts

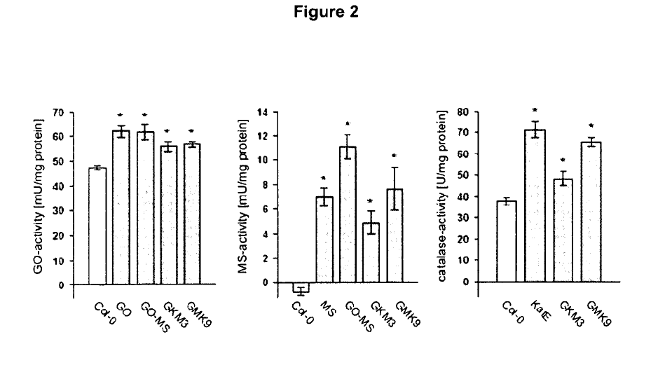

A. thaliana was transformed with a plasmid encoding glycolate oxidase (GO). Kanamycin-resistant transgenic lines with GO activities higher than the wild-type (19 to 53%) and containing only one GO transgene insertion were subjected to two more rounds of screening to obtain nonsegregating T3 transgenic lines. A GO representative line with a 30% higher GO activity compared to the wild-type (FIG. 2) was transformed with a plasmid encoding malate synthase (MS). Hygromycin-resistant GO-MS transformants were selected and analysed for MS activity. As all transgenic lines obtained showed similar MS activities (9.8±2.2 to 17.9±0.5 mU/mg protein), a representative line (FIG. 2) bearing only one MS transgene insertion as revealed by Southern blot, was subjected to two more rounds of screening to obtain a nonsegregating T3 population. This double homozygous overexpressing line was transformed with a plasmid encoding E. coli catalase (KatE) and eleven independent lines (GMK lines) were selected by BASTAresistance. GMK lines containing catalase activities higher than the wild-type were subjected to two more rounds of screening to obtain nonsegregating T3 lines. The lines GMK3 and GMK9 were selected for further analysis as representative of the two different populations obtained (see below; FIG. 2). Southern blot analysis indicated that the line GMK3 contained only one KatE transgene insertion while GMK9 contained three insertions (not shown). The GMK plants presented GO and MS activities that resembled the parental lines GO and GO-MS (FIG. 2), indicating no loss of transgene expression after nine generations. To exclude any phenotypic alteration caused by the single transgene expression, MS and KatE were also expressed in the wild-type background. In both cases, lines with MS or catalase activities similar to those found in the GMK lines were obtained (representative lines are included in FIG. 2). Enzymatic measurements performed with isolated chloroplasts from leaves of 5-week-old GMK3 plants and wildtype plants demonstrated that the transgene enzymes were correctly localized to plastids (not shown).

### Example 11

### Transgenic GMK Plants Accumulate More Biomass

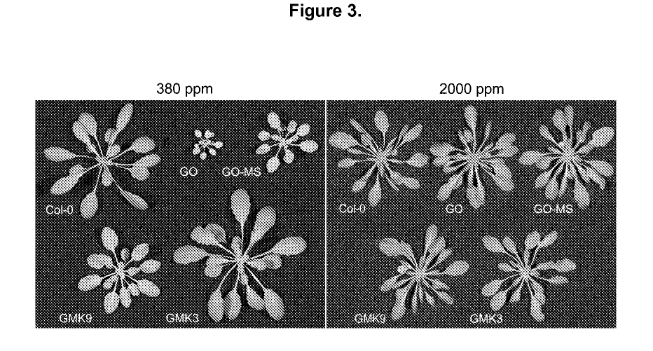

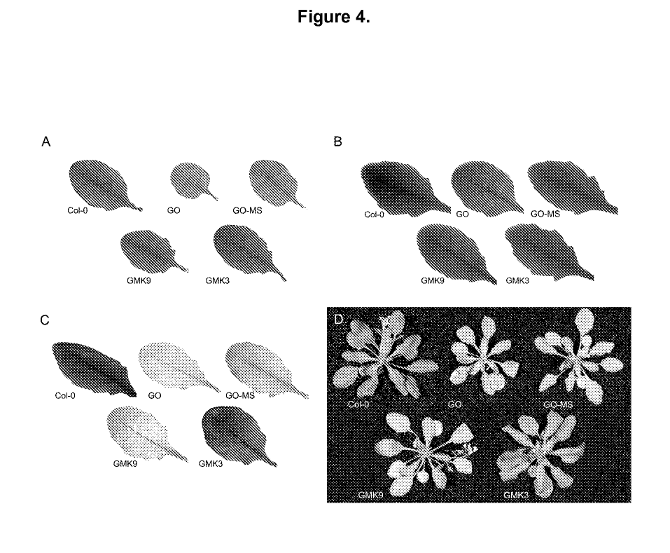

Homozygous GO plants were characterized by a reduced rosette diameter and fresh weight and yellowish leaves during the first seven weeks of growth (FIG. 3). Interestingly from the seventh week on, the differences in growth with respect to the wild-type became less pronounced (not shown). Measurements of glyoxylate and water peroxide contents indicated that both were increased in the GO plants (FIG. S1). The GO-MS lines resembled the GO ones but with an intermediate size between the GO and the wild-type plants (FIG. 3). As the GO-MS plants presented the GO phenotype when exposed to stress conditions (see below), the KatE gene was expressed in the plastids of the GO-MS plants to promote the detoxification of excess H₂O₂ produced by the GO activity. Independent lines presenting all three transgenic activities showed a statistically significant increase in biomass (e. g. GMK3) while others were bigger than the parental GO-MS plants but still smaller than the wild-type (e. g. GMK9). As shown in table 1, GMK3 plants showed an increased rosette diameter accompanied by a higher number of leaves and an increase in the dry weight by 35 to 40% compared to the wild-type. Negligible phenotypical differences were observed when all these lines were grown under non-photorespiratory conditions (CO2 concentration of 2000 ppm (FIG. 3 and Table 1). Obviously, the inhibition of the oxygenase reaction of RubisCO led to a reduced production of glycolate inside the chloroplast and, consequently, also to reduced glyoxylate and H₂O₂ levels. It is worth mentioning that the single MS and KatE overexpressing lines behaved like the wild-type under all conditions tested (not shown). We then evaluated the capacity of the transformants to accumulate the end products of photosynthesis at the end of the light period. Sucrose and the monosaccharides glucose and fructose were determined by GC-MS. At ambient CO₂ and in the absence of stress, the GO-MS plants accumulated less sucrose, while the GMK lines showed levels comparable to the wild-type. While no differences in the accumulation of glucose and fructose were observed in the GO-MS plants compared to the wild-type, the levels in GMK lines were higher than those of the wild-type. After exposure to six hours of high light, all these metabolites were decreased in the GO-MS plants, whilst GMK lines accumulated these photoassimilates (Table 2). Likewise, under normal light conditions the GO-MS transformants accumulated less transitory starch (also observed in the parental GO line), while the GMK plants recovered the capacity to accumulate starch as it is the case for the wild-type (FIG. 4A). Similar high levels of starch accumulation were observed in all lines when they were grown at high CO₂ concentration (FIG. 4B). Interestingly, when the lines were grown under normal conditions and then transferred for seven days to high light conditions, the GMK3 plants accumulated starch like the wild-type, while the GO, GO-MS and GMK9 transgenic lines have lost this capacity (FIG. 4D). In line with these results, the GO, GO-MS and GMK9 lines showed intense yellowing and oxidative lesions in response to high light treatment (FIG. 4D).

### Example 12

### Enhanced CO₂ Assimilation and Reduced Photorespiration in GMK Plants

To evaluate the photosynthetic performance of the transgenic plants, chlorophyll fluorescence, gas exchange and discrimination against ¹³O (carbon isotope ratio, δ¹³C) were determined in the different genotypes. The electron transport rate (ETR) of the GO plants was diminished by approximately 25% in comparison to the wild-type, while those of the GO-MS and GMK9 plants was similar to that of the wild-type (Table 3). In the case of the GMK3 plants, the ETR was enhanced by 26% compared to the wild-type (Table 3). The F_{V}/F_{M} ratio indicated that the maximum quantum efficiency of photosystem II was not affected in 7-week-old GO-MS and GMK plants grown under normal conditions, while the GO plants showed a slightly negative effect (Table 3). It is worth mentioning that younger GO plants always presented much lower F_{V}/F_{M} ratios than the other lines, a fact that correlated well with the more pronounced visual phenotype of these plants during the first weeks of growth (not shown). On the other hand, when the plants were exposed to high light for six hours, no differences in the F_{V}/F_{M} ratio was observed in the GMK lines compared to the wild-type, while a strong photoinhibition was observed in GO and GO-MS plants (not shown). Instantaneous photosynthetic rate measurements (A, μmol m⁻² s⁻¹) were made on both control and transformants expressing the whole pathways using a LICOR portable photosynthesis meter. Table 3 shows that the photosynthetic rate of the GMK3 line was relatively higher than the wild-type, while the one of line GMK9 was comparable to the wild-type. To determine if GMK3 plants are photosynthetically more efficient than their parental lines, carbon isotope fractionation studies were performed. Carbon isotope ratio measurements provide an integrated view of photosynthetic function performed of plants throughout their life span. As shown in Table 3, when plants were grown under normal conditions, the δ¹³C were less negative for the GMK3 line compared to the wild-type (a similar result was obtained for the GMK1 line), indicating increased carboxylation efficiency and water use efficiency in these plants. As anticipated, the δ¹³C of all lines showed no differential trend when the plants were grown under non-photorespiratory conditions (2,000 ppm CO₂, Table 3). Glycine and serine levels were determined by GC-MS and the Gly/Ser ratio was calculated as an indicator of flux through the photorespiratory pathway. The GMK lines presented lower glycine contents than the wild-type at ambient CO2 concentrations and under normal or high light conditions (Table 3). The GO-MS plants showed invariable glycine contents under normal light conditions but showed decreased levels under high light (Table 3). In the case of serine, the GMK plants showed lower levels than the wildtype under both light intensities tested, while the GO-MS plants showed wild-type levels under normal light and increased serine levels under high light (Table 3). The Gly/Ser ratio of all lines was comparable under normal light conditions but showed significantly lower ratios determined for the transgenic lines after six hours at high light intensities compared to the wild-type (Table 3). After incubating the plants under this photorespiratory condition, the Gly/Ser ratio of the wild-type increased about 150-fold compared to normal light conditions due to a high accumulation of glycine (Table 3). This increase was only 33, 13- and 63-fold in the case of GO-MS, GMK3 and GMK9, respectively (Table 3). The GMK3 plants presented the lowest Gly/Ser ratio due to a very low glycine accumulation in these plants. No differences in the Gly/Ser ratio were observed between the lines when grown at high CO₂ concentrations (Table 3).

TABLE 1
Growth parameters of 8-week-old transgenic and wild-type lines.

| | | wild-type | GO-MS | GMK3 | GMK9 |
|---|---|---|---|---|---|
| RD (cm) | A | 10.5 ± 0.2 | 9.4 ± 0.3 | 12.1 ± 0.2 | 9.4 ± 0.3 |
| NL | A | 33.3 ± 0.8 | 33.4 ± 0.5 | 39.5 ± 0.7 | 32.2 ± 0.9 |
| | E | 31.0 ± 0.4 | 32.0 ± 0.6 | 31.1 ± 0.6 | 32.1 ± 0.5 |
| DW (mg) | A | 106.9 ± 5.6 | 106.7 ± 6.0 | 146.6 ± 3.9 | 104.3 ± 8.3 |
| | E | 184.7 ± 16.1 | 185.4 ± 7.9 | 199.7 ± 8.4 | 199.6 ± 5.9 |

Rosette diameter (RD), number of leaves (NL) and dry weight (DW) of plants grown in short days at ambient (A, 380 ppm) or at elevated CO₂ (E, 2,000 ppm) concentrations. The values presented are the means ± SE of two replicates of pools of at least eight plants each. The values set in bold case indicate significant differences to the wild-type values calculated by the Student's test (P < 0.05).

TABLE 2
Photosynthate levels measured by GC-MS of whole rosette leaves harvested after 6 hours in the light
at different light intensities (L: low light (30 μmol m⁻² s⁻¹); H: high light (600 μmol m⁻² s⁻¹)).

| | wild-type | | GO-MS | | GMK3 | | GMK9 | |
|---|---|---|---|---|---|---|---|---|
| | L | H | L | H | L | H | L | H |
| Glucose | 8.43 ± 1.48 | 122.5 ± 4.6 | 11.49 ± 2.36 | 35.80 ± 0.50 | 12.13 ± 0.65 | 86.8 ± 8.6 | 10.87 ± 0.64 | 103.5 ± 2.5 |
| Fructose | 1.65 ± 0.25 | 10.30 ± 0.51 | 1.63 ± 0.18 | 3.27 ± 0.05 | 3.11 ± 0.03 | 8.83 ± 0.64 | 2.28 ± 0.18 | 16.27 ± 0.90 |
| Sucrose | 34.90 ± 1.90 | 338.7 ± 16.7 | 24.50 ± 2.30 | 160.9 ± 6.1 | 36.30 ± 2.70 | 173.5 ± 3.10 | 33.80 ± 0.70 | 236.2 ± 8.2 |
| Glycine | 0.67 ± 0.03 | 59.62 ± 7.20 | 0.68 ± 0.04 | 18.50 ± 0.90 | 0.51 ± 0.08 | 9.50 ± 1.90 | 0.35 ± 0.14 | 28.10 ± 5.10 |
| Serine | 16.80 ± 1.20 | 9.70 ± 1.40 | 18.80 ± 1.20 | 15.50 ± 0.40 | 6.70 ± 1.80 | 5.90 ± 0.50 | 9.00 ± 2.80 | 7.6 ± 1.50 |

The values presented are the means ± SE of two replicates of pools of at least eight plants each. The values set in bold case indicate significant differences to the wild-type values calculated by the Student's test (P < 0.05).

TABLE 3
Photosynthetic performance of wild-type and the transgenic lines.

| | Wild-type | GO | GO-MS | GMK3 | GMK9 |
|---|---|---|---|---|---|
| A (μmol m⁻² s⁻¹) | 8.23 ± 0.21 | n.d. | n.d. | 9.16 ± 0.17 | 8.72 ± 0.18 |
| ETR (μmol m⁻² s⁻¹) | 70.5 ± 2.7 | 52.9 ± 9.8 | 74.6 ± 4.1 | 89.1 ± 3.4 | 75.8 ± 2.6 |
| F_{V}/F_{M} | 0.68 ± 0.02 | 0.54 ± 0.02 | 0.55 ± 0.01 | 0.69 ± 0.04 | 0.65 ± 0.05 |
| Δδ¹³C | | | | | |
| at 380 ppm | | n.d. | n.d. | 0.87 ± 0.01 | −0.11 ± 0.07 |
| at 2,000 ppm | | n.d. | n.d. | −0.04 ± 0.02 | −0.19 ± 0.08 |
| Gly/Ser | | | | | |
| at 380 ppm (30 μmol m⁻² s⁻) | 0.040 ± 0.001 | 0.051 ± 0.015 | 0.037 ± 0.001 | 0.050 ± 0.011 | 0.032 ± 0.009 |
| at 380 ppm (600 μmol m⁻² s⁻) | 6.238 ± 0.206 | 1.235 ± 0.049 | 1.189 ± 0.039 | 1.574 ± 0.235 | 3.807 ± 0.382 |
| at 2,000 ppm | 0.035 ± 0.001 | 0.032 ± 0.001 | 0.039 ± 0.003 | 0.043 ± 0.004 | 0.040 ± 0.004 |

A, CO₂ assimilation rate at 380 ppm CO₂; ETR, Electron Transport Rate determined at 600 μmol m⁻² s⁻¹; F_{V}/F_{M}, maximum quantum efficiency of photosystem II determined after six hours at 600 μmol m⁻² s⁻¹; Δδ¹³C, Differences in whole leaf δ¹³C values of the transformants when compared to the wild-type; Gly/Ser, Glycine to serine ratio. The values presented are the means ± SE of two replicates of pools of at least eight plants each and those set in bold case indicate significant differences to the wild-type values calculated by the Student's test (P < 0.05). If not stated otherwise, the measurements were conducted with plants grown under normal light intensities. n.d.: not determined.

References as far as not mentioned explicitly in the description are found in the following list of references:

### REFERENCES

1. Tolbert N E (1997) The C2 oxidative photosynthetic carbon cycle. Annu. Rev. Plant Physiol. Plant Mol. Biol. 48: 1-25.
2. Douce R and Neuburger M (1999) Biochemical dissection of photorespiration. Curr. Opin. Plant Biol. 2: 214-222.
3. Wingler A, Lea P J, Quick W P, Leegood R C (2000) Photorespiration: metabolic pathways and their role in stress protection. Phil. Trans. R. Soc. Lond B 255: 1517-1529.
4. Keys A J, Bird I F, Cornelius M J, Lea P J, Wallsgrove R M, Miflin B J (1978) Photorespiratory nitrogen cycle. Nature 275: 741-743.
5. Leegood R C, Lea P J, Adcok M D, Häusler R E (1995) The regulation and control of photorespiration. J. Exp. Bot. 46: 1397-1414.
6. Parry M, Andralojc P C, Mitchell R A C, Madgwick P, Keys A (2003) Manipulation of RubisCO: the amount, activity, function and regulation. J. Exp. Bot. 54: 132-1333.
7. Somerville C R, Ogren W L (1980) Photorespiration mutants of Arabidopsis thaliana deficient in serine-glycolate aminotransferase activity. Proc. Natl. Acad. Sci. USA 77: 2684-2687.
8. Somerville C R, Ogren W L (1980) Inhibition of photosynthesis in Arabidopsis mutants lacking leaf glutamate synthase activity. Nature 286: 257-259.
9. Somerville C R, Ogren W L (1981) Photorespiration-deficient mutants of Arabidopsis thaliana lacking mitochondrial serine transhydroxymethylase activity. Plant Physiol. 67: 666-671.
10. Somerville C R, Ogren W L (1982) Mutants of the cruciferous plant Arabidopsis thaliana lacking glycine decarboxylase activity. Biochem. J. 202: 373-380. 25
11. Sommerville S, Somerville C R (1985) Mutant of Arabidopsis thaliana deficient in chloroplast dicarboxylate transport is missing an envelope protein. Plant Sci. Lett. 37: 217-220.
12. Bauwe H. and Kolukisaoglu U. (2003) Genetic manipulation of glycine decarboxylation. J Exp. Bot. 54: 1523-1535.
13. Bold R, Edner C, Kolukisaoglu U, Hagemann M, Weckwerth W, et al. (2005) DGlycerate 3-kinase the last unknown enzyme in the photorespiratory cycle in Arabidopsis, belongs to a novel kinase family. Plant Cell 17: 2413-2420.
14. Bagder M R, Price G D (2003) CO2 concentrating mechanisms in cyanobacteria: molecular components, their diversity and evolution. J. Exp. Bot. 54: 609-622.
15. Giordano M, Beardall J, Raven J A (2005) CO2 concentrating mechanisms in algae: mechanisms, environmental modulation and evolution. Ann. Rev. Plant Biol. 56, 99-131.
16. Hatch M D (1987) C4 photosynthesis: A unique bend of modified biochemistry, anatomy and ultrastructure. Biochim. Biophys. Acta 895: 8-16.
17. Sage R F, Pearcy R W, Seemann J R (1987) The nitrogen use efficiency of C3 and C4plants. Plant Physiol. 85: 355-359.
18. Ishimaru K, Ichikawa H, Matsuoka M, Oshugi R (1997) Analysis of a C4 pyruvate, orthophosphate dikinase expressed in C3 transgenic Arabidopsis plants. Plant Sci. 129: 57-64.
19. Ishimaru K, Ohkawa Y, Ishige T, Tobias D J, Oshugi R (1998) Elevated pyruvate orthophosphate dikinase (PPDK) activity alters carbon metabolism in C3-transgenic potatoes with a C4 maize PPDK gene. Physiol. Plant. 103: 340-346.
20. Matsuoka M, Furbank R T, Fukayama H, Miyao M (2001) Molecular engineering of C4 photosynthesis. Ann. Rev. Plant Physiol. Plant Mol. Biol. 52: 297-314.
21. Häusler R E, Rademacher T, Li J, Lipka V, Fischer K L, et al. (2001) Single and double overexpression of C4-cycle genes had differential effects on the pattern of endogenous enzymes, attenuation of photorespiration and on contents of UV protectants in transgenic potato and tobacco plants. J. Exp. Bot. 52: 1785-1803.
22. Kebeish R, Niessen N, Thiruveedhi K, Bari R, Hirsch J-H, et al. (2007) Chloroplastic photorespiratory bypass increases photosynthesis and biomass production in Arabidopsis thaliana. Nature Biotech. 25: 593-599.
23. Keys A J (1999) Biochemistry of photorespiration and the consequences for plant performance. In Plant Carbohydrate Biochemistry (eds. J A Bryant, M M Burell, N J Kruger), pp. 147-162. Oxford: BIOS Scientific Publishers.
24. Noctor G, Arisi A-C M, Jouanin L, Foyer C H (1999) Photorespiratory glycine enhances glutathione accumulation in both the chloroplastic and cytosolic compartments. J. Exp. Bot. 50: 1157-1167.
25. Heber U, Bligny R, Streb P, Douce R (1996) Photorespiration is essential for protection of the photosynthetic apparatus of C3-plants against photoinactivation under sunlight. Bot. Acta. 109: 307-315.
26. Kozaki A, Takeba G (1996) Photorespiration protects C3-plants from photooxidation. Nature 384, 557-560.
27. Farquhar G D, Ehleringer J R, Hubick K T (1989) Carbon isotope discrimination and photosynthesis. Ann. Rev. Plant Physiol. 40: 503-537.
28. Gillon J S, Griffiths H (1997) The influence of (photo)respiration on carbon isotope discrimination in plants. Plant Cell Environ. 20:1217-1230.
29. Igamberdiev A U, Mikkelsen T, Ambus P, Bauwe H, Lea P, et al. (2004) Photorespiration contributes to stomatal regulation and carbon isotope fractionation: a study with barley, potato and Arabidopsis plant deficient in glycine decarboxylase. Photos Res. 81: 139-152.
30. von Cammerer S (2003) C4 photosynthesis in a single cell is theoretically inefficient but may ameliorate internal CO2 diffusion limitations of C3 leaves. Plant Cell Environm. 26: 1191-1197.
31. Horng J-T, Behari R, Burke C-A, Baker A (1995) Investigation of the Energy Requirement and Targeting Signal for the Import of Glycolate Oxidase into Glyoxysomes. Eur. J. Biochem. 230: 157-163.
32. Mori H, Takeda-Yoshikawa Y, Hara-Nishimura I, Nishimura M (1991) Pumpkin malate synthase Cloning and sequencing of the cDNA and Northern blot analysis. Eur. J. Biochem. 197: 331-336.
33. Fahnenstich H, Saigo M, Niessen M, Zanor M I, et al. (2007) Alteration of organic acid metabolism in Arabidopsis thaliana overexpressing the maize C4-NADP-malic enzyme causes accelerated senescence during extended darkness. Plant Physiol. 145: 640-652.
34. Hellens R P, Edwards E A, Leyland N R, Bean S, Mullineaux P M (2000) pGreen: a versatile and flexible binary Ti vector for Agrobacterium-mediated plant transformation. Plant Mol. Biol. 42: 819-832.
35. Bechtold, N, Ellis J and Pelletier, G (1993) In planta Agrobacterium-mediated gene transfer by infiltration of adult Arabidopsis thaliana plants. C R Acad Sci Paris, Life Sci. 316: 1194-1199.
36. Yamaguchi K and Nishimura M (2000) reduction to below threshold levels of glycolate oxidase activities in transgenic tobacco enhances photoinhibition during irradiation. Plant Cell Physiol. 41: 1397-1406.
37. Smith C V, Huang C-C, Miczak A, Russell D G, Sacchettini J C, et al. (2003) Biochemical and Structural Studies of Malate Synthase from Mycobacterium tuberculosis. J. Biol. Chem. 278: 1735-1743.
38. Havir E, McHale N (1987) Biochemical and developmental characterization of multiple forms of catalase in tobacco leaves. Plant Physiol. 84: 450-455.
39. Okuda T, Matsuda Y, Yamanaska A, Sagisaka S (1991) Abrupt increase in the level of hydrogen peroxide in leaves of winter wheat is caused by cold treatment. Plant Physiol. 97: 1265-1267.
40. Häusler R E, Bailey K J, Lea P J, Leegood R C (1996) Control of photosynthesis in barley mutants with reduced activities of glutamine synthetase and glutamate synthase. Planta 200: 388-396.
41. Awada T, Dunigan D D, Dickman M B (2004) Animal anti-apoptotic genes enhance recovery from drought stress in tobacco. Int. J. Agri & Biol. 6: 943-949.
42. Madhavan S, Treichel I, O'Leary M H (1991) Effects of relative humidity on carbon isotope fractionation in plants. Bot. Acta 104: 292-294.

1. A plant cell comprising in its chloroplasts enzymatic activities for converting glycolate into malate.

2. The plant cell of claim 1, comprising in its chloroplasts a first polypeptide having glycolat oxidase activity, a second polypeptide having malate synthase activity and a third polypeptide having catalase activity.

3. The plant cell of claim 2, wherein said first polypeptide is encoded by a first polynucleotide comprising a nucleic acid selected from the group consisting of:
a. a nucleic acid having a nucleotide sequence as shown in SEQ ID NO: 1;
b. a nucleic acid encoding a polypeptide having an amino acid sequence as shown in SEQ ID NO: 2;
c. a nucleic acid having a nucleotide sequence being at least 50% identical to the nucleotide sequence shown in SEQ ID NO: 1, wherein said nucleic acid encodes a polypeptide having glycolate oxidase activity; and
d. a nucleic acid encoding a polypeptide having an amino acid sequence being at least 50% identical to the amino acid sequence shown in SEQ ID NO: 2, wherein said polypeptide has glycolate oxidase activity.

4. The plant cell of claim 3, wherein said second polypeptide is encoded by a second polynucleotide comprising a nucleic acid selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity.

5. The plant cell of claim 4, wherein said third polypeptide is encoded by a third polynucleotide comprising a nucleic acid selected from the group consisting of:
a. a nucleic acid having a nucleotide sequence as shown in SEQ ID NO: 5;
b. a nucleic acid encoding a polypeptide having an amino acid sequence as shown in SEQ ID NO: 6;
c. a nucleic acid having a nucleotide sequence being at least 70% identical to the nucleotide sequence shown in SEQ ID NO: 5, wherein said nucleic acid encodes a polypeptide having catalase activity; and
d. a nucleic acid encoding a polypeptide having an amino acid sequence being at least 50% identical to the amino acid sequence shown in SEQ ID NO: 6, wherein said polypeptide has catalase activity.

6. The plant cell of claim 3, wherein said first polypeptide is expressed from a heterologous polynucleotide.

7. The plant cell of claim 6, wherein said heterologous polynucleotide comprises a nucleic acid selected from the group consisting of:
a. a nucleic acid having a nucleotide sequence as shown in SEQ ID NO: 1;
b. a nucleic acid encoding a polypeptide having an amino acid sequence as shown in SEQ ID NO: 2;
c. a nucleic acid having a nucleotide sequence being at least 50% identical to the nucleotide sequence shown in SEQ ID NO: 1, wherein said nucleic acid encodes a polypeptide having glycolate oxidase activity; and
d. a nucleic acid encoding a polypeptide having an amino acid sequence being at least 50% identical to the amino acid sequence shown in SEQ ID NO: 2, wherein said polypeptide has glycolate oxidase activity.

8. The plant cell of claim 7, wherein said second polypeptide is expressed from a heterologous polynucleotide.

9. The plant cell of claim 8, wherein said heterologous polynucleotide comprises a nucleic acid selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity.

10. The plant cell of claim 9, wherein said third polypeptide is expressed from a heterologous polynucleotide.

11. The plant cell of claim 10, wherein said heterologous polynucleotide comprises a nucleic acid selected from the group consisting of:
a. a nucleic acid having a nucleotide sequence as shown in SEQ ID NO: 5;
b. a nucleic acid encoding a polypeptide having an amino acid sequence as shown in SEQ ID NO: 6;
c. a nucleic acid having a nucleotide sequence being at least 70% identical to the nucleotide sequence shown in SEQ ID NO: 5, wherein said nucleic acid encodes a polypeptide having catalase activity; and
d. a nucleic acid encoding a polypeptide having an amino acid sequence being at least 50% identical to the amino acid sequence shown in SEQ ID NO: 6, wherein said polypeptide has catalase activity.

12. The plant cell of claim 1, comprising in its chloroplasts a first polypeptide having glycolat dehydrogenase activity and a second polypeptide having malate synthase.

13. The plant cell of claim 12, wherein said first polypeptide is encoded by a first polynucleotide comprising a nucleic acid selected from the group consisting of:
a. a nucleic acid having a nucleotide sequence as shown in SEQ ID NO: 7;
b. a nucleic acid encoding a polypeptide having an amino acid sequence as shown in SEQ ID NO: 8;
c. a nucleic acid having a nucleotide sequence being at least 50% identical to the nucleotide sequence shown in SEQ ID NO: 7 wherein said nucleic acid encodes a polypeptide having glycolate dehydrogenase activity; and
d. a nucleic acid encoding a polypeptide having an amino acid sequence being at least 50% identical to the amino acid sequence shown in SEQ ID NO: 8, wherein said polypeptide has glycolate dehydrogenase activity.

14. The plant cell of claim 13, wherein said second polypeptide is encoded by a second polynucleotide comprising a nucleic acid selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity.

15. The plant cell of claim 14, wherein said first polypeptide is expressed from a heterologous polynucleotide.

16. The plant cell of claim 15, wherein said heterologous polynucleotide comprises a nucleic acid selected from the group consisting of:
a. a nucleic acid having a nucleotide sequence as shown in SEQ ID NO: 7;
b. a nucleic acid encoding a polypeptide having an amino acid sequence as shown in SEQ ID NO: 8;
c. a nucleic acid having a nucleotide sequence being at least 50% identical to the nucleotide sequence shown in SEQ ID NO: 7 wherein said nucleic acid encodes a polypeptide has glycolate dehydrogenase activity; and
d. a nucleic acid encoding a polypeptide having an amino acid sequence being at least 50% identical to the amino acid sequence shown in SEQ ID NO: 8, wherein said polypeptide has glycolate dehydrogenase activity.

17. The plant cell of claim 16, wherein said second polypeptide is expressed from a heterologous polynucleotide

18. The plant cell of claim 17, wherein said heterologous polynucleotide comprises a nucleic acid selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity.

19. The plant cell of claim 18, wherein said first, second and/or third polypeptide comprises a chloroplast transit peptide.

20. The plant cell of claim 19, wherein said plant cell further comprises an NADP-malic enzyme and a pyruvate dehydrogenase.

21. The plant cell of claim 20, wherein said plant cell has an increased efficiency of CO₂ assimilation compared to a plant cell lacking enzymatic activities for converting glycolate into malate.

22. The plant cell of claim 12, wherein said plant cell is a cell of a C₃ plant.

23. A plant comprising the plant cell of claim 1.

24. A plant comprising the plant cell of claim 1, wherein said plant has increased CO₂ assimilation compared to a plant lacking said plant cell.

25. The plant of claim 24, wherein said plant has an attenuated photorespiration compared to said plant cell.

26. The plant of claim 25, wherein said plant has increased water use efficiency compared to a plant lacking said plant cell.

27. The plant of claim 26, wherein said plant produces an increased number of leaves compared to a plant lacking said plant cell.

28. The plant of claim 27, wherein said plant has an increased yield compared to a plant lacking said plant cell.

29. A seed obtainable from the plant of claim 23.

30. A seed obtainable from a plant comprising the plant cell of claim 1, wherein said seed is capable of generating a plant comprising said plant cell.

31. A method for producing a transgenic plant or a plant cell having an increased water use efficiency compared to a corresponding non-transgenic plant or plant cell, said method comprises introducing in the chloroplasts of the transgenic plant or plant cell a first polypeptide having glycolat oxidase activity, a second polypeptide having malate synthase activity and a third polypeptide having catalase activity or the first polypeptide and the second polypeptide as defined in claim 13.

32. A method for producing a transgenic plant or plant cell having increased yield compared to a corresponding non-transgenic plant or plant cell, said method comprises generating in the chloroplasts of the transgenic plant or plant cell a first polypeptide having glycolat oxidase activity, a second polypeptide having malate synthase activity and a third polypeptide having catalase activity or the first polypeptide and the second polypeptide as defined in claim 13.

33. The method of claim 31, wherein said method comprises the steps of:
a. introducing at least one heterologous polynucleotide encoding said polypeptides into the plant of plant cell; and
b. expressing said polypeptides from said at least one polynucleotide.

34. A polynucleotide comprising the nucleic acid as defined in claim 3, in combination with a second nucleic acid and a third nucleic acid, wherein the second nucleic acid is selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity,
and
wherein the third nucleic acid is selected from the group consisting of:
a. a nucleic acid having a nucleotide sequence as shown in SEQ ID NO: 5;
b. a nucleic acid encoding a polypeptide having an amino acid sequence as shown in SEQ ID NO: 6;
c. a nucleic acid having a nucleotide sequence being at least 70% identical to the nucleotide sequence shown in SEQ ID NO: 5, wherein said nucleic acid encodes a polypeptide having catalase activity; and
d. a nucleic acid encoding a polypeptide having an amino acid sequence being at least 50% identical to the amino acid sequence shown in SEQ ID NO: 6, wherein said polypeptide has catalase activity.

35. A polynucleotide comprising the nucleic acid as defined in claim 13 in combination with a nucleic acid selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity.

36. A vector comprising the polynucleotide of claim 34.

37. The vector of claim 36, wherein said vector is an expression vector.

38. A composition comprising a first polynucleotide as defined in claim 3, a second polynucleotide and a third polynucleotide, wherein the second polynucleotide comprises a nucleic acid selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity,
and
wherein the third polynucleotide comprises a nucleic acid selected from the group consisting of:
a. a nucleic acid having a nucleotide sequence as shown in SEQ ID NO: 5;
b. a nucleic acid encoding a polypeptide having an amino acid sequence as shown in SEQ ID NO: 6;
c. a nucleic acid having a nucleotide sequence being at least 70% identical to the nucleotide sequence shown in SEQ ID NO: 5, wherein said nucleic acid encodes a polypeptide having catalase activity; and
d. a nucleic acid encoding a polypeptide having an amino acid sequence being at least 50% identical to the amino acid sequence shown in SEQ ID NO: 6, wherein said polypeptide has catalase activity.

39. A composition comprising a first polynucleotide as defined in claim 13 and a second polynucleotide comprising a nucleic acid selected from the group consisting of:
a. a nucleic acid having a nucleotide sequence as shown in SEQ ID NO: 3;
b. a nucleic acid encoding a polypeptide having an amino acid sequence as shown in SEQ ID NO: 4;
c. a nucleic acid having a nucleotide sequence being at least 40% identical to the nucleotide sequence shown in SEQ ID NO: 3, wherein said nucleic acid encodes a polypeptide having malate synthase activity; and
d. a nucleic acid encoding a polypeptide having an amino acid sequence being at least 40% identical to the amino acid sequence shown in SEQ ID NO: 4, wherein said polypeptide has malate synthase activity.

40-41. (canceled)


SEQUENCE LISTING (18 sequences)

SEQ ID NO: 1 - DNA, 1104 bp, Arabidopsis thaliana
atggagatcactaacgttaccgagtatgatgcaatcgcaaagcagaagctgcctaagatggtgtacgactactatgcatctggtgcagaagaccaatggactcttcaagagaacagaaacgcttttgcaaggatcctctttcggcctcggattctgattgatgtgagcaagattgacatgacaaccaccgtcttggggttcaagatctcgatgcccatcatggttgctccaactgccatgcaaaagatggctcaccctgatggggaatatgctactgctagagctgcatctgcagctggaactatcatgacactatcttcatgggctacttccagcgttgaagaagttgcgtctacagggccagggatccgattcttccagctctatgtatacaagaacaggaatgtggttgagcagctcgtgagaagagctgagagggctgggttcaaagccattgctctcactgtagacaccccaaggctaggccgcagagagtctgatatcaagaacagattcactttgcctccaaacctgacattgaagaactttgaaggacttgacctcggaaagatggacgaggccaatgactctggcttggcttcatatgttgctggtcaaattgaccgtaccttaagctggaaggatgtccagtggctccagacaatcaccaagttgcccattcttgtcaaaggtgttcttacaggagaggatgcaaggatagcgattcaagctggtgcagccggaatcattgtatcaaaccatggagctcgccagcttgactatgtcccagcaaccatctcggcccttgaagaggttgtcaaagcgacacaaggacgaattcctgtcttcttggatggtggtgttcgacgtggcactgatgtcttcaaagcacttgcacttggagcctccgggatatttattggaagaccagtggtattctcattggcagctgaaggagaggctggagttagaaaggtgcttcaaatgctacgtgatgagttcgagctgaccatggcactgagtgggtgtcggtccctaaaggaaatctcccgtaaccacattaccaccgaatgggacactccacgtccttcagccaggttatag

SEQ ID NO: 2 - protein, 367 aa, Arabidopsis thaliana
MEITNVTEYDAIAKQKLPKMVYDYYASGAEDQWTLQENRNAFARILFRPRILIDVSKIDMTTTVLGFKISMPIMVAPTAMQKMAHPDGEYATARAASAAGTIMTLSSWATSSVEEVASTGPGIRFFQLYVYKNRNVVEQLVRRAERAGFKAIALTVDTPRLGRRESDIKNRFTLPPNLTLKNFEGLDLGKMDEANDSGLASYVAGQIDRTLSWKDVQWLQTITKLPILVKGVLTGEDARIAIQAGAAGIIVSNHGARQLDYVPATISALEEVVKATQGRIPVFLDGGVRRGTDVFKALALGASGIFIGRPVVFSLAAEGEAGVRKVLQMLRDEFELTMALSGCRSLKEISRNHITTEWDTPRPSARL

SEQ ID NO: 3 - DNA, 1947 bp, Cucurbita pepo
atgggatcgctgggaatgtattctgaatcggcagtaaggaagaaaagtagccgaggctacgatgttccagagggagtggacattcggggacgttatgatgaagaatttgccaggattctcaacaaggaagccttgctgtttgtggctgatttacagaggactttcagaaaccacataaggtattcgatggaatgccgcagagaagccaaaaggaggtacaatgaaggggcggtgccggggtttgatccggcgaccaagtatataagggaatctgagtggacatgtgcatcagtccccccggcagttgctgatcggagagtggagatcaccggacctgtggagcggaagatgatcatcaacgcactcaattctggagctaaagttttcatggcggactttgaagatgcactatcaccaaactgggagaatttgatgagggggcaaattaatctgaaggatgcagttgatgggactataagcttccatgacaaagctagaaacaaggtttataaactgaacgatcagacagccaagctctttgttcgccctcgaggttggcacttcgctgaggctcatatcttcatcgacggcgagcctgccaccggctgtcttgtggacttcgggctctacttttttcacaaccatgccaatttccggcgctctcaaggtcaaggttctggccctttcttttaccttcccaaaatggagcactccagggaagcaaaaatatggaacagtgtatttgagagagcagagaagatggcagggatagagaggggcagcatcagggccactgtgctgattgaaacacttccagcagtgtttcaaatggatgaaatactctatgagctgagggatcattctgtgggattgaactgtggtagatgggattacatattcagctatgtcaagaccttccaggctcacctagatcgcctgttacccgaccgagtccaagtcggtatggcacaacatttcatgaggagttattctgatctccttatcaggacttgtcacacggtggtgtgccacgtgggaggcatggctgctcaaattccaattagagacgacccgaaggcaaatgagatggcacttgagctagtgaggaaggacaaattgagagaggcaaaggcaggacatgatggaacatgggcagcacatccaggattaatcccagcatgtatggaagtcttcaccaacagcatgggaaatgcccccaatcagatccgatctgcaagacgagacgatgctgcaaacctaactgaagacgacctcttgcagcaaccgaggggtgttcgtacattggaagggctccggttgaacacccgagtcggaattcagtacctagcagcatggctaaccgggacaggctctgtgcctctctacaaccttatggaagatgcagccacagctgaaatcagcagggttcaaaactggcaatggctgaagtatggagtggaattggatggagatgggcttggagtgagagtgaacaaggaactgttcgcaagagtggtggaagaagaaatggaaaggattgaaagagaagtggggaaggagaaattcaggaagggaatgtacaaagaggcttgcaagatgttcacaaggcaatgcacagcgccaaccttggatgattttctgaccttggatgcgtacaaccacatagtcatacatcatcccagggagctgtccaggctctgaaaaccgcaccaaattggccttctttcgacaggtaagagttcttttgcttcccttctcttctcttttcttctcttccttgttgctgagaataacgtatttgtattgtaatttaagactctgtttgtttgtccgtttctttttttaagctgtttgtgtcggttatgtcaagaaattcagcaattgcaagcaaattggaactctctccatgaacctaaaaaaaaaaaaaaaaaaaaaaaaaaaaaaaaa

SEQ ID NO: 4 - protein, 566 aa, Cucurbita pepo
MGSLGMYSESAVRKKSSRGYDVPEGVDIRGRYDEEFARILNKEALLFVADLQRTFRNHIRYSMECRREAKRRYNEGAVPGFDPATKYIRESEWTCASVPPAVADRRVEITGPVERKMIINALNSGAKVFMADFEDALSPNWENLMRGQINLKDAVDGTISFHDKARNKVYKLNDQTAKLFVRPRGWHFAEAHIFIDGEPATGCLVDFGLYFFHNHANFRRSQGQGSGPFFYLPKMEHSREAKIWNSVFERAEKMAGIERGSIRATVLIETLPAVFQMDEILYELRDHSVGLNCGRWDYIFSYVKTFQAHLDRLLPDRVQVGMAQHFMRSYSDLLIRTCHTVVCHVGGMAAQIPIRDDPKANEMALELVRKDKLREAKAGHDGTWAAHPGLIPACMEVFTNSMGNAPNQIRSARRDDAANLTEDDLLQQPRGVRTLEGLRLNTRVGIQYLAAWLTGTGSVPLYNLMEDAATAEISRVQNWQWLKYGVELDGDGLGVRVNKELFARVVEEEMERIEREVGKEKFRKGMYKEACKMFTRQCTAPTLDDFLTLDAYNHIVIHHPRELSRL

SEQ ID NO: 5 - DNA, 2262 bp, Escherichia coli
atgtcgcaacataacgaaaagaacccacatcagcaccagtcaccactacacgattccagcgaagcgaaaccggggatggactcactggcacctgaggacggctctcatcgtccagcggctgaaccaacaccgccaggtgcacaacctaccgccccagggagcctgaaagcccctgatacgcgtaacgaaaaacttaattctctggaagacgtacgcaaaggcagtgaaaattatgcgctgaccactaatcagggcgtgcgcatcgccgacgatcaaaactcactgcgtgccggtagccgtggtccaacgctgctggaagattttattctgcgcgagaaaatcacccactttgaccatgagcgcattccggaacgtattgttcatgcacgcggatcagccgctcacggttatttccagccatataaaagcttaagcgatattaccaaagcggatttcctctcagatccgaacaaaatcaccccagtatttgtacgtttctctaccgttcagggtggtgctggctctgctgataccgtgcgtgatatccgtggctttgccaccaagttctataccgaagagggtatttttgacttcgttggcaataacacgccaatcttctttatccaggatgcgcataaattccccgattttgttcatgcggtaaaaccagaaccgcactgggcaattccacaagggcaaagtgcccacgatactttctgggattatgtttctctgcaacctgaaactctgcacaacgtgatgtgggcgatgtcggatcgcggcatcccccgcagttaccgcaccatggaaggcttcggtattcacaccttccgcctgattaatgccgaagggaaggcaacgtttgtacgtttccactggaaaccactggcaggtaaagcctcactcgtttgggatgaagcacaaaaactcaccggacgtgacccggacttccaccgccgcgagttgtgggaagccattgaagcaggcgattttccggaatacgaactgggcttccagttgattcctgaagaagatgaattcaagttcgacttcgatcttctcgatccaaccaaacttatcccggaagaactggtgcccgttcagcgtgtcggcaaaatggtgctcaatcgcaacccggataacttctttgctgaaaacgaacaggcggctttccatcctgggcatatcgtgccgggactggacttcaccaacgatccgctgttgcagggacgtttgttctcctataccgatacacaaatcagtcgtcttggtgggccgaatttccatgagattccgattaaccgtccgacctgcccttaccataatttccagcgtgacggcatgcatcgcatggggatcgacactaacccggcgaattacgaaccgaactcgattaacgataactggccgcgcgaaacaccgccggggccgaaacgcggcggttttgaatcataccaggagcgcgtggaaggcaataaagttcgcgagcgcagcccatcgtttggcgaatattattcccatccgcgtctgttctggctaagtcagacgccatttgagcagcgccatattgtcgatggtttcagttttgagttaagcaaagtcgttcgtccgtatattcgtgagcgcgttgttgaccagctggcgcatattgatctcactctggcccaggcggtggcgaaaaatctcggtatcgaactgactgacgaccagctgaatatcaccccacctccggacgtcaacggtctgaaaaaggatccatccttaagtttgtacgccattcctgacggtgatgtgaaaggtcgcgtggtagcgattttacttaatgatgaagtgagatcggcagaccttctggccattctcaaggcgctgaaggccaaaggcgttcatgccaaactgctctactcccgaatgggtgaagtgactgcggatgacggtacggtgttgcctatagccgctacctttgccggtgcaccttcgctgacggtcgatgcggtcattgtcccttgcggcaatatcgcggatatcgctgacaacggcgatgccaactactacctgatggaagcctacaaacaccttaaaccgattgcgctggcgggtgacgcgcgcaagtttaaagcaacaatcaagatcgctgaccagggtgaagaagggattgtggaagctgacagcgctgacggtagttttatggatgaactgctaacgctgatggcagcacaccgcgtgtggtcacgcattcctaagattgacaaaattcctgcctga

SEQ ID NO: 6 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 7 - DNA, 1704 bp, Arabidopsis thaliana
atggctttcgcttcaaaattcgctcgttctaaaactattctctcttttctccggccttgtcgtcagctccactcgacgcccaagtcaaccggtgacgtgaccgtactttctccggtgaagggacgccggagacttccaacttgctggtcgagctctctgttcccattggcgatagctgcctccgccacttctttcgcttacctgaatctgtcgaatccttctatcagtgaatcatcatcggctttggattcaagagatataactgttggtggaaaagatagcactgaagctgttgttaaaggagaatacaaacaagtccctaaggagcttatttctcaattgaaaactatcctcgaggataacttgacaactgactacgatgagaggtacttccatgggaagccccagaacagttttcacaaggcagtcaacattcctgatgtcgttgttttccctaggtccgaagaagaagtctccaagattcttaaatcctgcaatgaatataaggttcctattgtaccatatggtggggcaacatcgatcgagggtcatacccttgctccaaaaggaggtgtgtgcattgacatgtcattaatgaagagggtgaaagcattacatgtggaggatatggatgttattgttgagcctggaattggttggctggagcttaatgaatatttggaagagtatggtctattctttcctcttgatccaggacctggtgcctccataggaggcatgtgtgctacgcgttgctctggctctttagctgtaaggtatggaactatgcgtgacaatgttataagcctcaaggtggttcttcctaatggagatgttgtgaagacagcttcacgtgccagaaagagtgctgctggatacgatttgactcgcttgataattgggagtgagggtactttaggagtcattactgagattactctccgacttcagaaaatcccacagcattcagtggtggcagtttgcaatttccctacagttaaggatgctgcagacgtggccattgccactatgatgtctggaatacaggtgtcaagagtggaactccttgacgaggttcaaatcagagctattaatatggcaaacgggaaaaatttgactgaagctccaactctgatgttcgagtttataggaacagaggcatatacacgtgagcagacgcaaattgttcagcaaattgcttccaaacacaatggatcagactttatgttcgcagaagaacctgaagcaaaaaaagaactctggaagataagaaaagaggcgctgtgggcttgctatgctatggcgccaggtcatgaagcaatgattacagatgtctgtgtccctttatctcaccttgcagaactcatatcaagatccaaaaaagagcttgatgcatcatcgttgttgtgtaccgttattgctcatgccggagatggaaactttcacacatgtattatgtttgatccaagcagtgaagagcagagaagagaagcagaaagactgaaccactttatggttcacagtgcactgtccatggatggaacatgtactggagaacacggtgttggaacaggaaaaatgaagtatctggagaaggaactgggaatagaagcactgcaaactatgaagagaatcaagaaaacgttggacccaaacgatatcatgaacccgggaaagttaattcctcctcatgtatgtttctaa

SEQ ID NO: 8 - protein, 567 aa, Arabidopsis thaliana
MAFASKFARSKTILSFLRPCRQLHSTPKSTGDVTVLSPVKGRRRLPTCWSSSLFPLAIAASATSFAYLNLSNPSISESSSALDSRDITVGGKDSTEAVVKGEYKQVPKELISQLKTILEDNLTTDYDERYFHGKPQNSFHKAVNIPDVVVFPRSEEEVSKILKSCNEYKVPIVPYGGATSIEGHTLAPKGGVCIDMSLMKRVKALHVEDMDVIVEPGIGWLELNEYLEEYGLFFPLDPGPGASIGGMCATRCSGSLAVRYGTMRDNVISLKVVLPNGDVVKTASRARKSAAGYDLTRLIIGSEGTLGVITEITLRLQKIPQHSVVAVCNFPTVKDAADVAIATMMSGIQVSRVELLDEVQIRAINMANGKNLTEAPTLMFEFIGTEAYTREQTQIVQQIASKHNGSDFMFAEEPEAKKELWKIRKEALWACYAMAPGHEAMITDVCVPLSHLAELISRSKKELDASSLLCTVIAHAGDGNFHTCIMFDPSSEEQRREAERLNHFMVHSALSMDGTCTGEHGVGTGKMKYLEKELGIEALQTMKRIKKTLDPNDIMNPGKLIPPHVCF

SEQ ID NO: 9 - DNA, 30 bp, Artificial sequence
  primer GO fow
tacaattggagatcactaacgttaccgagt

SEQ ID NO: 10 - DNA, 33 bp, Artificial sequence
  primer GO rev1
tgggacactccacgtccttagtctagactagta

SEQ ID NO: 11 - DNA, 34 bp, Artificial sequence
  primer MS fow
acaccggtcgctgggaatgtattctgaatcggca

SEQ ID NO: 12 - DNA, 36 bp, Artificial sequence
  primer MS rev1
cacataggcatacatcatcccaggtgagtcgacgtt

SEQ ID NO: 13 - DNA, 31 bp, Artificial sequence
  primer KatE fow1
acaccggtcgcaacataacgaaaagaaccca

SEQ ID NO: 14 - DNA, 31 bp, Artificial sequence
  primer KatE rev1
acgtcgactcaggcaggaattttgtcaatct

SEQ ID NO: 15 - DNA, 32 bp, Artificial sequence
  primer PGM fow
taggtacccaatcaacaatgacgtcgacctac

SEQ ID NO: 16 - DNA, 32 bp, Artificial sequence
  primer PGM rev
gagattaaatcgttgccgacgaagcaattgta

SEQ ID NO: 17 - DNA, 30 bp, Artificial sequence
  primer rbcS3C fow
acgagctcatccagaattggcgttggatta

SEQ ID NO: 18 - DNA, 33 bp, Artificial sequence
  primer rbcS3C rev
agcaacggtggaagagtcagttgcaaccggtat